(19) United States
(12) Patent Application Publication
(10) Pub. No.: US 2024/0027433 A1
(43) Pub. Date: Jan. 25, 2024

(54) STRUCTURE AND METHODS FOR DETECTION OF SAMPLE ANALYTES

(71) Applicant: SOMALOGIC OPERATING CO., INC., Boulder, CO (US)

(72) Inventors: Ashwin GOPINATH, Boulder, CO (US); Paul ROTHEMUND, Boulder, CO (US); Rishabh SHETTY, Boulder, CO (US); Shane BOWEN, Boulder, CO (US)

(21) Appl. No.: 18/245,131

(22) PCT Filed: Sep. 14, 2021
(86) PCT No.: PCT/US21/50262

§ 371 Date: Mar. 13, 2023

Related U.S. Application Data
(60) Provisional application No. 63/078,837, filed on Sep. 15, 2020.

(51) Int. Cl. G01N 33/53

(52) U.S. Cl. CPC G01N 33/5308

(57) ABSTRACT

Provided herein are structures and methods for detecting one or more analyte molecules present in a sample. In some embodiments, the one or more analyte molecules are detected using one or more supramolecular structures. In some embodiments, the one or more supramolecular structures are specifically designed to minimize cross-reactivity with each other. In some embodiments, the supramolecular structures are bi-stable, wherein the supramolecular structures shift from an unstable state to a stable state through interaction with one or more analyte molecules from the sample. In some embodiments, the stable state supramolecular structures are configured to provide a signal for analyte molecule detection and quantification. In some embodiments, the signal correlates to a DNA signal, such that detection and quantification of an analyte molecule comprises converting the presence of the analyte molecule into a DNA signal.

### CROSS-REFERENCE

This application claims the benefit of U.S. Provisional Patent Application No. 63/078,837, filed Sep. 15, 2020, which is incorporated by reference herein in its entirety.

### BACKGROUND

The current state of personalized healthcare is overwhelmingly genome-centric, predominantly focused on quantifying the genes present within an individual. While such an approach has proven to be extremely powerful, it does not provide a clinician with the complete picture of an individual's health. This is because genes are the “blueprints” of an individual and it merely informs the likelihood of developing an ailment. Within an individual these “blueprints” first need to be transcribed into RNA and then translated into various protein molecules, the real “actors” in the cell, in order to have any effect on the health of an individual.

The concentration of proteins, the interaction between the proteins (protein-protein interactions or PPI), as well as the interaction between proteins and small molecules, are intricately linked to the health of different organs, homeostatic regulatory mechanism as well as the interaction of these systems with the external environment. Hence, quantitative information about proteins as well as PPIs is vital to create a complete picture of an individual's health at a given time point as well as to predict any emerging health issues. For instance, the amount of stress experienced by cardiac muscles (e.g. during a heart attack) can be inferred by measuring the concentration of troponin I/II and myosin light chain present within peripheral blood. Similar protein biomarkers have also been identified, validated and are deployed for a wide variety of organ dysfunctions (e.g. liver disease and thyroid disorders), specific cancers (e.g. colorectal or prostate cancer), and infectious diseases (e.g. HIV and Zika). The interaction between these proteins are also essential for drug development and are increasingly becoming a highly sought-after dataset. The ability to detect and quantify proteins and other molecules, within a given sample of bodily fluids, is an integral component of such healthcare development.

### SUMMARY

The present disclosure generally relates to systems, structures and methods for detection and quantification of analyte molecules in a sample.

Provided herein, in some embodiments, is a method for detecting an analyte molecule present in a sample, the method comprising: a) providing a supramolecular structure comprising: i) a core structure comprising a plurality of core molecules, ii) a capture molecule linked to the core structure at a first location, and iii) a detector molecule linked to the core structure at a second location, wherein the supramolecular structure is in an unstable state, such that the detector molecule is configured to be unbound from the core structure through cleavage of a link therebetween at the second location; b) contacting the sample with the supramolecular structure, such that the supramolecular structure shifts from the unstable state to a stable state wherein the detector molecule and the capture molecule are linked together through binding to the analyte molecule, thereby forming a link between the detector molecule and capture molecule; c) providing a trigger to cleave the link between the detector molecule and the core structure at the second location, wherein the detector molecule remains linked to the core structure through the link with the capture molecule; and d) detecting the analyte molecule based on a signal provided by the supramolecular structure that shifted to the stable state.

Provided herein, in some embodiments, is a method for detecting one or more analyte molecules present in a sample, the method comprising: a) providing a plurality of supramolecular structures, each comprising: i) a core structure comprising a plurality of core molecules, ii) a capture molecule linked to the core structure at a first location, and iii) a detector molecule linked to the core structure at a second location, wherein the supramolecular structure is in an unstable state, such that the detector molecule is configured to be unbound from the core structure through cleavage of a link therebetween at the second location; b) contacting the sample with the plurality of supramolecular structures, such that at least one supramolecular structure shifts from the unstable state to a stable state wherein the corresponding detector molecule and capture molecule are linked together through binding to an analyte molecule of the one or more analyte molecules, thereby forming a link between the corresponding detector molecule and capture molecule; c) providing a trigger to cleave the link between each detector molecule and corresponding core structure at the second location of the plurality of supramolecular structures, wherein the detector molecule for the at least one supramolecular structure that shifted to a stable state remains linked to the corresponding core structure through the link with the corresponding capture molecule; and d) detecting a respective analyte molecule of the one or more analyte molecules based on a signal provided by a respective supramolecular structure of the at least one supramolecular structures that shifted to the stable state. In some embodiments, the method further comprises isolating the plurality of supramolecular structures from any detector molecules unbound from any supramolecular structures that did not shift to a stable state.

In some embodiments, any method disclosed herein further comprising quantifying the concentration of the analyte molecule in the sample. In some embodiments, any method disclosed herein further comprising identifying the detected analyte molecule. In some embodiments, any method disclosed herein further comprising detecting the analyte molecule based on the signal when the analyte molecule is present in the sample at a count of a single molecule or higher. In some embodiments, for any method disclosed herein, the sample comprises a complex biological sample and the method provides for single-molecule sensitivity thereby increasing a dynamic range and quantitative capture of a range of molecular concentrations within the complex biological sample. In some embodiments, for any method disclosed herein, the analyte molecule comprises a protein, a peptide, a peptide fragment, a lipid, a DNA, a RNA, an organic molecule, an inorganic molecule, complexes thereof, or any combinations thereof. In some embodiments, for any method disclosed herein, each supramolecular structure is a nanostructure.

In some embodiments, for any method disclosed herein, each core structure is a nanostructure. In some embodiments, for any method disclosed herein, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, for any method disclosed herein, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

In some embodiments, for any method disclosed herein, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof.

In some embodiments, for any method disclosed herein, the respective analyte molecule is 1) bound to the capture molecule of the respective supramolecular structure through a chemical bond and/or 2) bound to the detector molecule of the respective supramolecular structure through a chemical bond. In some embodiments, for any method disclosed herein, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof. In some embodiments, for any method disclosed herein, wherein for each supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, for any method disclosed herein, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker.

In some embodiments, for any method disclosed herein, each supramolecular structure in the unstable state comprises the respective capture molecule and detector molecule spaced apart at a pre-determined distance, so as to reduce or inhibit the occurrence of cross-reactions between capture and/or detector molecules of a first supramolecular structure and corresponding capture and/or detector molecules of a second supramolecular structure. In some embodiments, for any method disclosed herein, the pre-determined distance is from about 3 nm to about 40 nm.

In some embodiments, for any method disclosed herein, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, wherein the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, for any method disclosed herein, the signal comprises the detector barcode, the capture barcode, or combinations thereof, corresponding to a supramolecular structure that shifted to a stable state. In some embodiments, any method disclosed herein, further comprising separating each detector barcode from a corresponding detector molecule for the at least one supramolecular structure that shifted to a stable state, such that the corresponding signal comprises the respective detector barcode for detection of the analyte molecule bound to the respective capture and detector molecules. In some embodiments, each separated detector barcode provides a DNA signal corresponding to the analyte molecule bound to the respective detector molecule. In some embodiments, the at least one separated detector barcodes are analyzed using genotyping, qPCR, sequencing, or combinations thereof. In some embodiments, a plurality of analyte molecules in the sample are detected simultaneously through multiplexing via one or more supramolecular structures that shifted to a stable state. In some embodiments, for any method disclosed herein, the capture and detector molecules for each supramolecular structure is configured for binding to one or more specific types of analyte molecules.

In some embodiments, for any method comprising using a plurality of supramolecular structures disclosed herein, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures.

In some embodiments, for any method comprising using a plurality of supramolecular structures disclosed herein, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure. In some embodiments, the plurality of supramolecular structures are attached to one or more widgets, one or more solid supports, one or more polymer matrices, one or more solid substrates, one or more molecular condensates, or combinations thereof. In some embodiments, the mean distance between any two supramolecular structures is larger than the pre-determined distance between the capture and detector molecules of a respective supramolecular structure. In some embodiments, each polymer matrix of the one or more polymer matrices comprises a hydrogel bead. In some embodiments, one or more supramolecular substrates are attached to a hydrogel bead. In some embodiments, each supramolecular structure is co-polymerized with the hydrogel bead through a corresponding anchor molecule linked to the respective core structure of the corresponding supramolecular structure. In some embodiments, the one or more supramolecular structures are embedded within the hydrogel bead. In some embodiments, each hydrogel bead is contacted with a single cell in the sample for intracellular analyte molecule detection at a single cell resolution. In some embodiments, each solid substrate of the one or more solid substrates comprises a microparticle. In some embodiments, one or more supramolecular substrates are attached to a solid surface of the microparticle. In some embodiments, the microparticle comprises a polystyrene particle, silica particle, magnetic particle, or paramagnetic particle. In some embodiments, each solid substrate is contacted with a single cell in the sample for intracellular analyte molecule detection at a single cell resolution. In some embodiments, each solid substrate of the one or more solid substrates comprises a planar substrate. In some embodiments, a plurality of supramolecular structures are disposed on the planar substrate, wherein the planar substrate comprises a plurality of binding sites, wherein each binding site is configured to link with a corresponding supramolecular structure. In some embodiments, the plurality of supramolecular structures are configured to detect the same analyte molecule. In some embodiments, for any method comprising using a planar substrate, further comprising providing a plurality of signaling elements configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures. In some embodiments, for any method comprising using a planar substrate, further comprising barcoding each supramolecular structure so as to identify the location of each supramolecular structure on the planar substrate. In some embodiments, for any method comprising using a planar substrate, further comprising providing a plurality of signaling elements configured to link with the detector molecules of the at least one supramolecular structure that shifted to the stable state. In some embodiments, each signaling element comprises a fluorescent molecule or microbead, a fluorescent polymer, highly charged nanoparticles or polymer.

In some embodiments, for any method disclosed herein, the sample comprises a biological particle or a biomolecule. In some embodiments, for any method disclosed herein, the sample comprises an aqueous solution comprising a protein, a peptide, a fragment of a peptide, a lipid, DNA, RNA, an organic molecule, a viral particle, an exosome, an organelle, or any complexes thereof. In some embodiments, for any method disclosed herein, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

Provided herein, in some embodiments, is a substrate for detecting one or more analyte molecules in a sample, the substrate comprising a plurality of supramolecular structures, each supramolecular structure comprising: a) a core structure comprising a plurality of core molecules, b) a capture molecule linked to the supramolecular core at a first location, and c) a detector molecule linked to the supramolecular core at a second location, wherein the supramolecular structure is in an unstable state, such that the detector molecule is configured to be unbound from the core structure through cleavage of a link therebetween at the second location; wherein each supramolecular structure is configured to shift from the unstable state to a stable state through interaction between the detector molecule, the capture molecule, and a respective analyte molecule of the one or more analyte molecules; wherein, upon interaction with a trigger, a respective supramolecular structure that shifted to the stable state provides a signal for detecting the respective analyte molecule.

In some embodiments, wherein upon interaction with the trigger, each detection molecule linked to a supramolecular structure in the unstable state becomes unbound from said supramolecular structure. In some embodiments, each core structure of the plurality of supramolecular structures is identical to each other. In some embodiments, the mean distance between any two supramolecular structures is larger than the pre-determined distance between the capture and detector molecules of a respective supramolecular structure. In some embodiments, the substrate comprises a solid support, solid substrate, a polymer matrix, or a molecular condensate. In some embodiments, the sample comprises a complex biological sample and the method provides for single-molecule sensitivity thereby increasing a dynamic range and quantitative capture of a range of molecular concentrations within the complex biological sample. In some embodiments, the one or more analyte molecules comprises a protein, a peptide, a peptide fragment, a lipid, a DNA, a RNA, an organic molecule, an inorganic molecule, complexes thereof, or any combinations thereof. In some embodiments, each supramolecular structure is a nanostructure. In some embodiments, each core structure is a nanostructure. In some embodiments, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof. In some embodiments, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof. In some embodiments, the respective analyte molecule is 1) bound to the capture molecule through a chemical bond and/or 2) bound to the detector molecule through a chemical bond. In some embodiments, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof.

In some embodiments, wherein for each supramolecular structure of the substrate: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, wherein the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker. In some embodiments, each supramolecular structure in the unstable state comprises the respective capture molecule and detector molecule spaced apart at a pre-determined distance, so as to reduce or inhibit the occurrence of cross-reactions between capture and/or detector molecules of a first supramolecular structure and corresponding capture and/or detector molecules of a second supramolecular structure. the pre-determined distance is from about 3 nm to about 40 nm.

In some embodiments, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, the signal comprises the detector barcode, the capture barcode, or combinations thereof, corresponding to a supramolecular structure that shifted to a stable state. In some embodiments, each detector barcode from a corresponding detector molecule for the at least one supramolecular structure that shifted to a stable state is configured to be separated from said corresponding detector molecule, such that the corresponding signal comprises the respective detector barcode for detection of the analyte molecule bound to said corresponding detector molecule. In some embodiments, each separated detector barcode provides a DNA signal corresponding to the analyte molecule bound to the respective detector molecule. In some embodiments, the at least one separated detector barcode is configured to be analyzed using genotyping, qPCR, sequencing, or combinations thereof. In some embodiments, one or more supramolecular structures are configured for multiplexing the sample, wherein a plurality of analyte molecules in the sample are detected simultaneously. In some embodiments, the capture and detector molecules for each supramolecular structure is configured for binding to one or more specific types of analyte molecules.

In some embodiments, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures. In some embodiments, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure.

In some embodiments, each substrate comprises a widget, a solid support, a polymer matrix, a solid substrate, or a molecular condensate. In some embodiments, the mean distance between any two supramolecular structures is larger than the pre-determined distance between the capture and detector molecules of a respective supramolecular structure. In some embodiments, the polymer matrix comprises a hydrogel bead. In some embodiments, one or more supramolecular substrates are attached to the hydrogel bead. In some embodiments, each supramolecular structure is co-polymerized with the hydrogel bead through a corresponding anchor molecule linked to the respective core structure of the corresponding supramolecular structure. In some embodiments, the one or more supramolecular structures are embedded within the hydrogel bead. In some embodiments, each hydrogel bead is configured to be contacted with a single cell in the sample for intracellular analyte molecule detection at a single cell resolution. In some embodiments, the solid substrate comprises a microparticle. In some embodiments, one or more supramolecular substrates are attached to a solid surface of the microparticle. In some embodiments, the microparticle comprises a polystyrene particle, silica particle, magnetic particle, or paramagnetic particle. In some embodiments, each solid substrate is configured to be contacted with a single cell in the sample for intracellular analyte molecule detection at a single cell resolution. In some embodiments, the solid substrate comprises a planar substrate. In some embodiments, a plurality of supramolecular structures are disposed on the planar substrate, wherein the planar substrate comprises a plurality of binding sites, wherein each binding site is configured to link with a corresponding supramolecular structure. In some embodiments, the plurality of supramolecular structures are configured to detect the same analyte molecule. In some embodiments, a plurality of signaling elements are configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures.

In some embodiments, the sample comprises a biological particle or a biomolecule. In some embodiments, the sample comprises an aqueous solution comprising a protein, a peptide, a fragment of a peptide, a lipid, DNA, RNA, an organic molecule, a viral particle, an exosome, an organelle, or any complexes thereof. In some embodiments, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

Provided herein, in some embodiments, is a supramolecular structure for detecting an analyte molecule in a sample, the supramolecular structure comprising: a) a core structure comprising a plurality of core molecules, b) a capture molecule linked to the supramolecular core at a first location, and c) a detector molecule linked to the supramolecular core at a second location, wherein the supramolecular structure is in an unstable state, such that the detector molecule is configured to be unbound from the core structure through cleavage of a link therebetween at the second location; wherein the supramolecular structure is configured to shift from the unstable state to a stable state through interaction between the detector molecule, the capture molecule, and an analyte molecule; wherein, upon interaction with a trigger, the supramolecular structure that shifted to the stable state provides a signal for detecting the analyte molecule.

In some embodiments, the sample comprises a complex biological sample and the method provides for single-molecule sensitivity thereby increasing a dynamic range and quantitative capture of a range of molecular concentrations within the complex biological sample. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the supramolecular structure is a nanostructure. In some embodiments, the core structure is a nanostructure. In some embodiments, the plurality of core molecules for the core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, the core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof. In some embodiments, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof. In some embodiments, the analyte molecule is 1) bound to the capture molecule through a chemical bond and/or 2) bound to the detector molecule through a chemical bond. In some embodiments, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof.

In some embodiments, where for the supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker. In some embodiments, the supramolecular structure in the unstable state comprises the respective capture molecule and detector molecule spaced apart at a pre-determined distance, so as to reduce or inhibit the occurrence of cross-reactions between capture and/or detector molecules of the supramolecular structure with corresponding capture and/or detector molecules of another supramolecular structure. In some embodiments, the pre-determined distance is from about 3 nm to about 40 nm.

In some embodiments, the supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, the signal comprises the detector barcode, the capture barcode, or combinations thereof, corresponding to a supramolecular structure that shifted to a stable state. In some embodiments, the detector barcode from a corresponding detector molecule for a supramolecular structure that shifted to a stable state is configured to be separated from said corresponding detector molecule, such that the corresponding signal comprises the respective detector barcode for detection of the analyte molecule bound to said corresponding detector molecule. In some embodiments, the separated detector barcode provides a DNA signal corresponding to the analyte molecule bound to the respective detector molecule. In some embodiments, the separated detector barcode is configured to be analyzed using genotyping, qPCR, sequencing, or combinations thereof. In some embodiments, the capture and detector molecules for the supramolecular structure is configured for binding to one or more specific types of analyte molecules.

In some embodiments, the supramolecular structure comprises a prescribed shape, size, molecular weight, or combinations thereof, so as to reduce or eliminate cross-reactions with another supramolecular structure. In some embodiments, the supramolecular structure comprises a plurality of capture and detector molecules. In some embodiments, the supramolecular structure comprises a prescribed stoichiometry of the capture and detector molecules so as to reduce or eliminate cross-reactions with another supramolecular structure.

In some embodiments, the sample comprises a biological particle or a biomolecule. In some embodiments, the sample comprises an aqueous solution comprising a protein, a peptide, a fragment of a peptide, a lipid, DNA, RNA, an organic molecule, a viral particle, an exosome, an organelle, or any complexes thereof. In some embodiments, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 depicts an exemplary depiction of a supramolecular structure and the related subcomponents.

FIG. 2 depicts an exemplary depiction of an assembled three-arm nucleic acid junction based supramolecular structure and related subcomponents.

FIG. 3 depicts an exemplary depiction of the individual subcomponents of the three-arm nucleic acid junction based supramolecular structure from FIG. 2.

FIG. 4 depicts an exemplary depiction of the deconstructor molecules corresponding to the subcomponents of the three-arm nucleic acid junction based supramolecular structure from FIG. 2.

FIG. 5 depicts an exemplary depiction of an assembled DNA origami based supramolecular structure and related subcomponents.

FIG. 6 depicts an exemplary depiction of the individual subcomponents of the DNA origami based supramolecular structure from FIG. 5.

FIG. 7 depicts an exemplary depiction of the deconstructor molecules corresponding to the subcomponents of the DNA origami based supramolecular structure from FIG. 5.

FIG. 8 provides an exemplary depiction of a supramolecular structure in an unstable state before and after being subject to a trigger (e.g., interaction with a deconstructor molecule).

FIG. 9 provides an exemplary depiction of a supramolecular structure in a stable state before and after being subject to a trigger (e.g., interaction with a deconstructor molecule).

FIG. 10 provides an exemplary depiction of a supramolecular structure shifting from an unstable state to a stable state after interaction with an analyte molecule, and the respective configurations before and after being subject to a trigger (e.g., interaction with a deconstructor molecule).

FIG. 11 provides an exemplary depiction of a supramolecular structure shifting from a stable state to an unstable state after interaction with an analyte molecule, and the respective configurations before and after being subject to a trigger (e.g., interaction with a deconstructor molecule).

FIG. 12 provides an exemplary depiction of a method for detecting and quantifying analyte molecules using a plurality of supramolecular structures.

FIG. 13 provides an exemplary depiction of a method for forming a hydrogel bead attached with a plurality of supramolecular structures.

FIG. 14 provides an exemplary depiction of a method for forming a hydrogel bead attached with a plurality of supramolecular structures, using droplet technology.

FIG. 15 provides an exemplary depiction of attaching a plurality of supramolecular structures onto a solid substrate (e.g., microparticle).

FIG. 16 provides an exemplary depiction of a method for detecting and quantifying analyte molecules using a plurality of supramolecular structures embedded within hydrogel beads.

FIG. 17 provides an exemplary depiction of trapping a single cell and supramolecular structures embedded within a hydrogel bead in a droplet, as part of a method for detecting and quantifying intracellular analyte molecules.

FIG. 18 provides an exemplary depiction of collecting and processing droplets enclosing a single cell and supramolecular structures embedded within a hydrogel bead, as part of a method for detecting and quantifying intracellular analyte molecules.

FIG. 19 provides an exemplary depiction of trapping supramolecular structures with captured intracellular analyte molecules (from FIG. 18) and barcoded beads in a droplet, as part of a method for detecting and quantifying intracellular analyte molecules.

FIG. 20 provides an exemplary depiction of collecting and processing droplets enclosing supramolecular structures with captured intracellular analyte molecules (from FIG. 18) and barcoded beads in a droplet, as part of a method for detecting and quantifying intracellular analyte molecules.

FIG. 21 provides an exemplary depiction of a method for detecting and quantifying analyte molecules using a plurality of supramolecular structures attached to a planar substrate.

FIG. 22 provides an exemplary depiction of DNA strands (S1, S2, and Core) and DNA assemblies (W1, W2, W3, W4, W5, W6, and W7) to make two exemplary DNA Widgets comprising a 3 part (3pt) Widget and a 5 part (5pt) Widget.

FIG. 23 provides an exemplary depiction of DNA components (S1, S2, and Core) to make a basic 3 part Widget.

FIG. 24 provides an exemplary depiction of a working principle of Bridge, Release, 3pt Widget, and 5pt Widget.

FIG. 25 provides an exemplary depiction of an agarose gel for demonstration of the working principle as shown in FIG. 24.

### DETAILED DESCRIPTION

Disclosed herein are structures and methods for detecting one or more analyte molecules present in a sample. In some embodiments, the one or more analyte molecules are detected using one or more supramolecular structures. In some embodiments, the one or more supramolecular structures are specifically designed to minimize cross-reactivity with each other. In some embodiments, the supramolecular structures are bi-stable, wherein the supramolecular structures shift from an unstable state to a stable state through interaction with one or more analyte molecules from the sample. In some embodiments, the stable state supramolecular structures are configured to provide a signal for analyte molecule detection and quantification. In some embodiments, the signal correlates to a DNA signal, such that detection and quantification of an analyte molecule comprises converting the presence of the analyte molecule into a DNA signal.

### Sample

In some embodiments, the sample comprises an aqueous solution comprising protein, peptides, peptide fragments, lipids, DNA, RNA, organic molecules, inorganic molecules, complexes thereof, or any combinations thereof. In some embodiments, the analyte molecules in the sample comprise protein, peptides, peptide fragments, lipids, DNA, RNA, organic molecules, inorganic molecules, complexes thereof, or any combinations thereof. In some embodiments, the analyte molecules comprise intact proteins, denatured proteins, partially or fully degraded proteins, peptide fragments, denatured nucleic acids, degraded nucleic acid fragments, complexes thereof, or combinations thereof. In some embodiments, the sample is obtained from tissue, cells, the environment of tissues and/or cells, or combinations thereof. In some embodiments, the sample comprises tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. In some embodiments, the sample is isolated from a primary source such as cells, tissue, bodily fluids (e.g., blood), environmental samples, or combinations thereof, with or without purification. In some embodiments, the cells are lysed using a mechanical process or other cell lysis methods (e.g., lysis buffer). In some embodiments, the sample is filtered using a mechanical process (e.g., centrifugation), micron filtration, chromatography columns, other filtration methods, or combinations thereof. In some embodiments, the sample is treated with one or more enzymes to remove one or more nucleic acids or one or more proteins. In some embodiments, the sample comprises intact proteins, denatured proteins, partially or fully degraded proteins, peptide fragments, denatured nucleic acids or degraded nucleic acid fragments. In some embodiments, the sample is collected from one or more individual persons, one or more animals, one or more plants, or combinations thereof. In some embodiments, the sample is collected from an individual person, animal and/or plant having a disease or disorder that comprises an infectious disease, an immune disorder, a cancer, a genetic disease, a degenerative disease, a lifestyle disease, an injury, a rare disease, an age-related disease, or combinations thereof.

### Supramolecular Structure

In some embodiments, the supramolecular structure is a programmable structure that can spatially organize molecules. In some embodiments, the supramolecular structure comprises a plurality of molecules linked together. In some embodiments, the plurality of molecules of the supramolecular structure interact with at least some of each other. In some embodiments, the supramolecular structure comprises a specific shape. In some embodiments, the supramolecular nanostructure comprises a prescribed molecular weight based on the plurality of molecules of the supramolecular structure. In some embodiments, the supramolecular structure is a nanostructure. In some embodiments the plurality of molecules is linked together through a bond, a chemical bond, a physical attachment, or combinations thereof. In some embodiments, the supramolecular structure comprises a large molecular entity, of specific shape and molecular weight, formed from a well-defined number of smaller molecules interacting specifically with each other. In some embodiments, the structural, chemical, and physical properties of the supramolecular structure are explicitly designed. In some embodiments, the supramolecular structure comprises a plurality of subcomponents that are spaced apart according to a prescribed distance. In some embodiments, at least a portion of the supramolecular structure is rigid. In some embodiments, at least a portion of the supramolecular structure is semi-rigid. In some embodiments, at least a portion of the supramolecular structure is flexible.

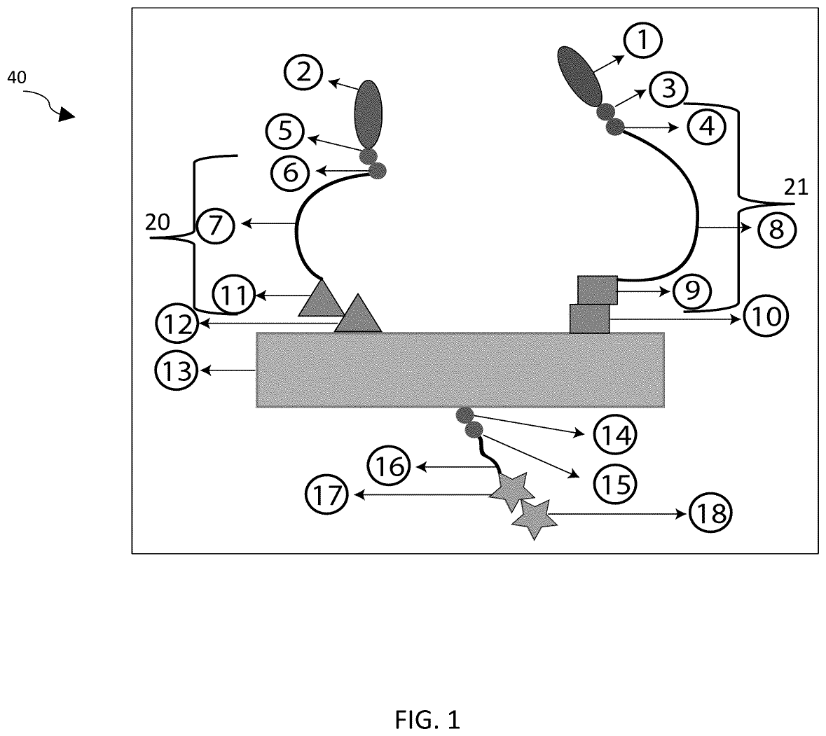

FIG. 1 provides an exemplary embodiment of a supramolecular structure 40 comprising a core structure 13, a capture molecule 2, a detector molecule 1, and an anchor molecule 18. In some embodiments, the supramolecular structure comprises one or more capture molecules 2, and one or more detector molecules 1 and optionally one or more anchor molecules 18. In some embodiments, the supramolecular structure does not comprise an anchor molecule. In some embodiments, the supramolecular structure is a polynucleotide structure.

In some embodiments, the core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises from about 2 unique molecules to about 1000 unique molecules. In some embodiments, the one or more core molecules interact with each other and define the specific shape of the supramolecular structure. In some embodiments, the plurality of core molecules interacts with each other through reversible non-covalent interactions. In some embodiments, the specific shape of the core structure is a three-dimensional (3D) configuration. In some embodiments, the one or more core molecules provide a specific molecular weight. In some embodiments, the core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the one or more nucleic acid strands comprise a single stranded scaffold strand and more than two staple strands. In some embodiments, the core structure comprises a polynucleotide structure. In some embodiments, at least a portion of the core structure is rigid. In some embodiments, at least a portion of the core structure is semi-rigid. In some embodiments, at least a portion of the core structure is flexible. In some embodiments, the core structure comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprising a DNA origami, RNA origami, or hybrid DNA/RNA origami that comprises a prescribed two-dimensional (2D) or 3D shape.

As used herein, the term “are linked together” in some embodiments refers to enabling the formation of a chemical bond. In some embodiments, as used herein, a chemical bond refers to a lasting attraction between atoms, ions or molecules. The bond includes covalent bonds, ionic bonds, hydrogen bonds, van der Waals interactions, or any combination thereof. In some embodiments, the term “are linked together” refers to hybridization of nucleic acids which is the process of combining two complementary single-stranded DNA or RNA molecules and allowing them to form a single double-stranded molecule through base pairing.

As used herein, the term “nucleic acid origami” generally refers to a nucleic acid construct comprising an engineered tertiary (e.g., folding and relative orientation of secondary structures) or quaternary structure (e.g., hybridization between strands that are not covalently linked to each other) in addition to the naturally-occurring secondary structure (e.g., helical structure) of nucleic acid(s). A nucleic acid origami may include DNA, RNA, PNA, modified or non-natural nucleic acids, or combinations thereof. A nucleic acid origami can include a “scaffold strand”. The scaffold strand can be circular (i.e., lacking a 5′ end and 3′ end) or linear (i.e., having a 5′ end and/or a 3′ end). A nucleic acid origami may include a plurality of oligonucleotides (“staple strands”) that hybridize via sequence complementarity to produce the engineered structuring of the origami particle. For example, the oligonucleotides can hybridize to a scaffold strand and/or to other oligonucleotides. A nucleic acid origami may comprise sections of single-stranded or double-stranded nucleic acid, or combinations thereof. Exemplary nucleic acid origami structures may include nanotubes, nanowires, cages, tiles, nanospheres, blocks, and combinations thereof. In some embodiments, the DNA origami comprises both single stranded and double stranded regions.

As shown in FIG. 1, in some embodiments, the core structure 13 is configured to be linked to a capture molecule 2, a detector molecule 1, an anchor molecule 18, or combinations thereof. In some embodiments, the capture molecule 2, detector molecule 1, and/or anchor molecule 18 are immobilized with respect to the core nanostructure 13 when linked thereto. In some embodiments, any number of the one or more core molecules comprises one or more core linkers 10,12,14 configured to form a linkage with a capture molecule 2, a detector molecule 1, and/or an anchor molecule 18. In some embodiments, any number of the one or more core molecules are configured to be linked with one or more core linkers 10,12,14 that are configured to form a linkage with a capture molecule 2, a detector molecule 1, and/or an anchor molecule 18. In some embodiments, one or more core linkers are linked to one or more core molecules through a chemical bond. In some embodiments, at least one of the one or more core linkers comprises a core reactive molecule. In some embodiments, each core reactive molecule independently comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain. In some embodiments, one or more core linkers comprise at least one extended staple strands which particularly protrude from the core structure. In some embodiments, the extended staple strands can be conjugated with (make a chemical bond with) a core reactive molecule. In some embodiments, the location of the extended staple strand is pre-determined.

In some embodiments, the core structure 13 is linked to 1) a capture molecule 2 at a prescribed first location on the core structure, 2) a detector molecule 1 at a prescribed second location on the core structure, and optionally 3) an anchor molecule 18 at a prescribed third location on the core structure. In some embodiments, a specified first core linker 12 is disposed at the first location on the core structure, and a specified second core linker 10 is disposed at the second location on the core structure. In some embodiments, one or more core molecules at the first location are modified to form a linkage with the first core linker 12. In some embodiments, the first core linker 12 is an extension of the core structure 13. In some embodiments, the first core linker 12 is an extended staple strand which particularly protrude from the core structure 13. In some embodiments, one or more core molecules at the second location is modified to form a linkage with the second core linker 10. In some embodiments, the second core linker 10 is an extension of the core structure 13. In some embodiments, the second core linker 10 is an extended staple strand which particularly protrude from the core structure 13. In some embodiments, the 3D shape of the core structure 13 and relative distances of the first and second locations are specified to maximize the intramolecular interactions between the capture molecule 2 and detector molecule 1. In some embodiments, the 3D shape of the core structure 13 and relative distances of the first and second locations are specified to obtain a desired distance between the capture molecule 2 and detector molecule 1, so as to maximize the intramolecular interactions between the capture molecule 2 and detector molecule 1.

As described herein, in some embodiments, the distance between the capture molecule 2 and detector molecule 1 is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance between the capture molecule 2 and detector molecule 1 is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about 40 nm to about 60 nm, including increments therein. In some embodiments, the distance between the capture molecule 2 and detector molecule 1 is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about nm. In some embodiments, the distance between the capture molecule 2 and detector molecule 1 is at most about 2 nm, about 5 nm, about 10 nm, about 20 nm, about 40 nm, or about nm.

As described herein, in some embodiments, the distance between the first location (corresponding to the capture molecule 2) and the second location (corresponding to the detector molecule 1) is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance the first location (corresponding to the capture molecule 2) and the second location (corresponding to the detector molecule 1) is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about 20 nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about nm to about 60 nm, including increments therein. In some embodiments, the distance between the first location (corresponding to the capture molecule 2) and the second location (corresponding to the detector molecule 1) is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about 40 nm. In some embodiments, the distance between the capture molecule 2 and detector molecule 1 the first location (corresponding to the capture molecule 2) and the second location (corresponding to the detector molecule 1) is at most about 2 nm, about 5 nm, about 10 nm, about 20 nm, about 40 nm, or about 60 nm.

In some embodiments, a specified third core linker 14 is disposed at the third location on the core structure 13. In some embodiments, one or more core molecules at the third location is modified to form a linkage with the third core linker 14. In some embodiments, the third core linker 14 is an extension of the core structure 13. In some embodiments, the first and second locations are disposed on a first side of the core structure 13, and the optional third location is disposed on a second side of the core structure 13. In some embodiments, the third core linker 14 comprises at least one extended staple strands which particularly protrude from the core structure 13. In some embodiments, the extended staple strands can be conjugated with (make a chemical bond with) a core reactive molecule. In some embodiments, the location of the extended staple strand is pre-determined.

In some embodiments, the capture molecule 2 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the detector molecule 1 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the anchor molecule comprises a reactive molecule. In some embodiments, the anchor molecule 18 comprises a reactive molecule. In some embodiments, the anchor molecule 18 comprises a DNA strand comprising a reactive molecule. In some embodiments, the anchor molecule 18 comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the anchor molecule 18 comprises a protein, a peptide, an antibody, an aptamers (RNA and DNA), a flourophore, a nanobody, a darpin, a catalyst, a polymerization initiator, a polymer like PEG, an organic molecule or combinations thereof. In some embodiments, a single pair of a capture molecule 2 and corresponding detector molecule 1 is linked to the core structure 13. In some embodiments, a plurality of pairs of capture molecules 2 and corresponding detector molecules 1 are linked to a core structure 13. In some embodiments, the plurality of pairs of capture molecules 2 and corresponding detector molecules 1 are spaced apart from each other to minimize cross-talk, i.e. minimizing capture and/or detector molecules from a first pair interacting with capture and/or detector molecules from a second pair.

In some embodiments, each component of the supramolecular structure may be independently modified or tuned. In some embodiments, modifying one or more of the components of the supramolecular structure may modify the 2D and 3D geometry of the supramolecular structure itself. In some embodiments, modifying one or more of the components of the supramolecular structure may modify the 2D and 3D geometry of the core structure. In some embodiments, such capability for independently modifying the components of the supramolecular nanostructure enables precise control over the organization of one or more supramolecular structures on solid surfaces (e.g., planar surfaces or microparticles) and 3D volumes (e.g., within a hydrogel matrix).

### Capture Barcode

As shown in FIG. 1, in some embodiments, the capture molecule 2 is linked to the core structure 13 through a capture barcode 20. In some embodiments, the capture barcode 20 forms a linkage with the capture molecule 2, and the capture barcode 20 forms a linkage with the core structure 13. In some embodiments, the capture barcode 20 comprises a first capture linker 11, a second capture linker 6, and a capture bridge 7. In some embodiments, the first capture linker 11 comprises a reactive molecule. In some embodiments, the first capture linker 11 comprises a reactive molecule comprising an amine, a thiol, a DBCO, a NHS ester, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the first capture linker 11 comprises a DNA sequence domain. In some embodiments, the DNA sequence domain is complementary to a DNA sequence domain of the first core linker 12. In some embodiments, the second capture linker 6 comprises a reactive molecule. In some embodiments, the second capture linker 6 comprises a reactive molecule comprising an amine, a thiol, a DBCO, a NHS ester, biotin, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the second capture linker comprises a DNA sequence domain. In some embodiments, the DNA sequence domain is complementary to DNA sequence domain of a third capture linker 5. In some embodiments, the capture bridge 7 comprises a polymer. In some embodiments, the capture bridge 7 comprises a polymer that comprises a nucleic acid (e.g., DNA or RNA) of a specific sequence. In some embodiments, the capture bridge 7 comprises a polymer such as PEG. In some embodiments, the first capture linker 11 is attached to the capture bridge 7 at a first terminal end thereof, and the second capture linker 6 is attached to the capture bridge 7 at a second terminal end thereof. In some embodiments, the first capture linker 11 is attached to the capture bridge 7 via a chemical bond. In some embodiments, the second capture linker 6 is attached to the capture bridge 7 via a chemical bond. In some embodiments, the first capture linker 11 is attached to the capture bridge 7 via a physical attachment. In some embodiments, the second capture linker 6 is attached to the capture bridge 7 via a physical attachment.

In some embodiments, the capture barcode 20 is linked to the core structure 13 through a linkage between the first capture linker 11 and the first core linker 12. In some embodiments, as described herein, the first core linker 12 is disposed at a first location on the core structure 13. In some embodiments, the first capture linker 11 and first core linker 12 are linked together through a chemical bond. In some embodiments, the first capture linker 11 and first core linker 12 are linked together through a covalent bond. In some embodiments, the first capture linker 11 and the first core linker 12 are linked together through hybridization between single stranded nucleic acids. In some embodiments, the linkage between the first capture linker 11 and first core linker 12 is reversible upon being subjected to a trigger. In some embodiments, the trigger comprises interaction with a deconstructor molecule (“capture deconstructor molecule”, e.g., reference character 30 in FIGS. 4,7) or exposure to a trigger signal. In some embodiments, the capture deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the capture barcode 20 is linked to the capture molecule 2 through a linkage between the second capture linker 6 and a third capture linker 5 that is bound to the capture molecule 2. In some embodiments, the third capture linker 5 comprises a reactive molecule. In some embodiments, the third capture linker 5 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third capture linker 5 comprises a DNA sequence domain. In some embodiments, the specific DNA sequence domains of the third capture linker 5 and the second linker 6 are complementary to each other. In some embodiments, the capture molecule 2 is bound to the third capture linker 5 through a chemical bond. In some embodiments, the capture molecule 2 is bound to the third capture linker 5 through a covalent bond. In some embodiments, the second capture linker 6 and third capture linker 5 are linked together through a chemical bond. In some embodiments, the second linker 6 and third capture linker 5 are linked together through a covalent bond. In some embodiments, the third capture linker 5 and the second capture linker 6 are linked together through hybridization between single stranded nucleic acids. In some embodiments, the linkage between the second capture linker 6 and third capture linker 5 is reversible upon being subjected to a trigger. In some embodiments, the trigger comprises interaction with a deconstructor molecule (“capture barcode release molecule”, e.g., reference character 31 in FIGS. 4,7) or exposure to a trigger signal. In some embodiments, the capture barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first capture linker 11 and first core linker 12 only, thereby breaking the capture molecule linkage with the core nanostructure 13 at the first location. In some embodiments, the capture barcode 20, when separated from the core structure 13 and the capture molecule 2, is configured to provide a signal for detecting an analyte molecule. In some embodiments, the signal as provided from the capture barcode 20 is a DNA signal.

As used herein, the term “DNA signal” in some embodiments refers to any change of the core nanostructure or a specific DNA sequence which may be identified by a nucleic acid sequencing process (for e.g., a capture barcode, detector barcode).

### Detector Barcode

As shown in FIG. 1, in some embodiments, the detector molecule 1 is linked to the core structure 13 through a detector barcode 21. In some embodiments, the detector barcode 21 forms a linkage with the detector molecule 1, and the detector barcode 21 forms a linkage with the core structure 13. In some embodiments, the detector barcode comprises a first detector linker 9, a second detector linker 4, and a detector bridge 8. In some embodiments, the first detector linker 9 comprises a reactive molecule. In some embodiments, the first detector linker 9 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first detector linker 9 comprises a DNA sequence domain. In some embodiments, the DNA sequence domain is complementary to DNA sequence domain of the second core linker 10. In some embodiments, the second detector linker 4 comprises a reactive molecule. In some embodiments, the second detector linker 4 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second detector linker 4 comprises a DNA sequence domain. In some embodiments, the DNA sequence domain is complementary to DNA sequence domain of a third detector linker 3. In some embodiments, the detector bridge 8 comprises a polymer. In some embodiments, the detector bridge 8 comprises a polymer that comprises a nucleic acid (DNA or RNA) of a specific sequence. In some embodiments, the detector bridge 8 comprises a polymer such as PEG. In some embodiments, the first detector linker 9 is attached to the detector bridge 8 at a first terminal end thereof, and the second detector linker 4 is attached to the detector bridge 8 at a second terminal end thereof. In some embodiments, the first detector linker 9 is attached to the detector bridge 8 via a chemical bond. In some embodiments, the second detector linker 4 is attached to the detector bridge 8 via a chemical bond. In some embodiments, the first detector linker 9 is attached to the detector bridge 8 via a physical attachment. In some embodiments, the second detector linker 4 is attached to the detector bridge 8 via a physical attachment.

In some embodiments, the detector barcode 21 is linked to the core structure 13 through a linkage between the first detector linker 9 and the second core linker 10. In some embodiments, as described herein, the second core linker 10 is disposed at a second location on the core structure 13. In some embodiments, the first detector linker 9 and second core linker 10 are linked together through a chemical bond. In some embodiments, the first detector linker 9 and second core linker 10 are linked together through a covalent bond. In some embodiments, the first detector linker 9 and second core linker 10 are linked together through hybridization between single stranded nucleic acids. In some embodiments, the linkage between the first detector linker 9 and second core linker 10 is reversible upon being subjected to a trigger. In some embodiments, the trigger comprises interaction with a deconstructor molecule (“detector deconstructor molecule”, e.g., reference character 28 in FIGS. 4,7) or exposure to a trigger signal. In some embodiments, the detector deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the detector barcode 21 is linked to the detector molecule 1 through a linkage between the second detector linker 4 and a third detector linker 3 bound to the detector molecule 1. In some embodiments, the third detector linker 3 comprises a reactive molecule. In some embodiments, the third detector linker 3 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third detector linker 3 comprises a DNA sequence domain. In some embodiments, the specific DNA sequence domains of the third detector linker 3 and the second detector linker 4 are complementary to each other. In some embodiments, the detector molecule 1 is bound to the third detector linker 3 through a chemical bond. In some embodiments, the detector molecule 1 is bound to the third detector linker 3 through a covalent bond. In some embodiments, the second detector linker 4 and third detector linker 3 are linked together through a chemical bond. In some embodiments, the second detector linker 4 and third detector linker 3 are linked together through a covalent bond. In some embodiments, the third detector linker 3 and the second detector linker 4 are linked together through hybridization between single stranded nucleic acids. In some embodiments, the linkage between the second detector linker 4 and third detector linker 3 is reversible upon being subjected to a trigger. In some embodiments, the trigger comprises interaction with a deconstructor molecule (“detector barcode release molecule”, e.g., reference character 29 in FIGS. 4,7) or exposure to a trigger signal. In some embodiments, the detector barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first detector linker 9 and second core linker 10 only, thereby breaking the detector molecule linkage with the core structure 13 at the second location. In some embodiments, the detector barcode 21, when separated from the core structure 13 and the detector molecule 2, is configured to provide a signal for detecting an analyte molecule. In some embodiments, the signal as provided from the detector barcode 21 is a DNA signal.

### Anchor Barcode

As shown in FIG. 1, in some embodiments, the anchor molecule 18 is linked to the core structure 13 through an anchor barcode. In some embodiments, the anchor barcode forms a linkage with the anchor molecule 18, and the anchor barcode forms a linkage with the core structure 13. In some embodiments, the anchor barcode comprises a first anchor linker 15, a second anchor linker 17, and an anchor bridge 16. In some embodiments, the first anchor linker 15 comprises a reactive molecule. In some embodiments, the first anchor linker 15 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first anchor linker 15 comprises a DNA sequence domain. In some embodiments, the DNA sequence domain is complementary to DNA sequence domain of the third core linker 14. In some embodiments, the second anchor linker 17 comprises a reactive molecule. In some embodiments, the second anchor linker 17 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second anchor linker 17 comprises a DNA sequence domain. In some embodiments, the anchor bridge 16 comprises a polymer. In some embodiments, the anchor bridge 16 comprises a polymer that comprises a nucleic acid (DNA or RNA) of a specific sequence. In some embodiments, the anchor bridge 16 comprises a polymer such as PEG. In some embodiments, the first anchor linker 15 is attached to the anchor bridge 16 at a first terminal end thereof, and the second anchor linker 17 is attached to the anchor bridge 16 at a second terminal end thereof. In some embodiments, the first anchor linker 15 is attached to the anchor bridge 16 via a chemical bond. In some embodiments, the second anchor linker 17 is attached to the anchor bridge 16 via a physical attachment. In some embodiments, the first anchor linker 15 is attached to the anchor bridge 16 via a chemical bond. In some embodiments, the second anchor linker 17 is attached to the anchor bridge 16 via a physical attachment.

In some embodiments, the anchor barcode is linked to the core structure 13 through a linkage between the first anchor linker 15 and the third core linker 14. In some embodiments, as described herein, the third core linker 14 is disposed at a third location on the core structure 13. In some embodiments, the first anchor linker 15 and third core linker 14 are linked together through a chemical bond. In some embodiments, the first anchor linker 15 and third core linker 14 are linked together through a covalent bond. In some embodiments, the first anchor linker 15 and third core linker 14 are linked together through hybridization between single stranded nucleic acids. In some embodiments, the linkage between the first anchor linker 15 and third core linker 14 is reversible upon being subjected to a trigger. In some embodiments, the trigger comprises interaction with a deconstructor molecule (“anchor deconstructor molecule”, e.g., reference character 32 in FIGS. 4,7) or exposure to a trigger signal. In some embodiments, the anchor deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the anchor barcode is linked to the anchor molecule 18 through a linkage between the second anchor linker 17 and the anchor molecule 18. As disclosed herein, in some embodiments, the anchor molecule comprises a reactive molecule, a DNA sequence domain, a DNA sequence domain comprising a reactive molecule, or combinations thereof. In some embodiments, the DNA sequences domain of the second anchor linker 17 and the anchor molecule 18 are complementary to each other. In some embodiments, the anchor molecule 18 is bound to the second anchor linker 17 through a chemical bond. In some embodiments, the anchor molecule 18 is bound to the second anchor linker 17 through a covalent bond. In some embodiments, the second anchor linker 17 and the anchor molecule 18 are linked together through hybridization between single stranded nucleic acids. In some embodiments, the linkage between the second anchor linker 17 and anchor molecule 18 is reversible upon being subjected to a trigger. In some embodiments, the trigger comprises interaction with a deconstructor molecule (“anchor barcode release molecule” e.g., reference character 33 in FIGS. 4,7) or exposure to a trigger signal. In some embodiments, the anchor barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first anchor linker 15 and third core linker 14 only, thereby breaking the anchor molecule linkage with the core structure 13 at the third location.

In some embodiments, the capture deconstructor molecule, capture barcode release molecule, detector deconstructor molecule, and detector barcode release molecule comprise the same type of molecule. In some embodiments, the capture deconstructor molecule, capture barcode release molecule, detector deconstructor molecule, and detector barcode release molecule comprise different types of molecules. In some embodiments, the capture deconstructor molecule, capture barcode release molecule, detector deconstructor molecule, detector barcode release molecule, anchor deconstructor molecule, and anchor barcode release molecule comprise the same type of molecules. In some embodiments, the capture deconstructor molecule, capture barcode release molecule, detector deconstructor molecule, detector barcode release molecule, anchor deconstructor molecule, and anchor barcode release molecule comprise different types of molecules. In some embodiments, any combination of the capture deconstructor molecule, capture barcode release molecule, detector deconstructor molecule, detector barcode release molecule, anchor deconstructor molecule, and anchor barcode release molecule comprise the same type of molecules.

### Three Arm Nucleic Acid Junction Based Supramolecular Structure

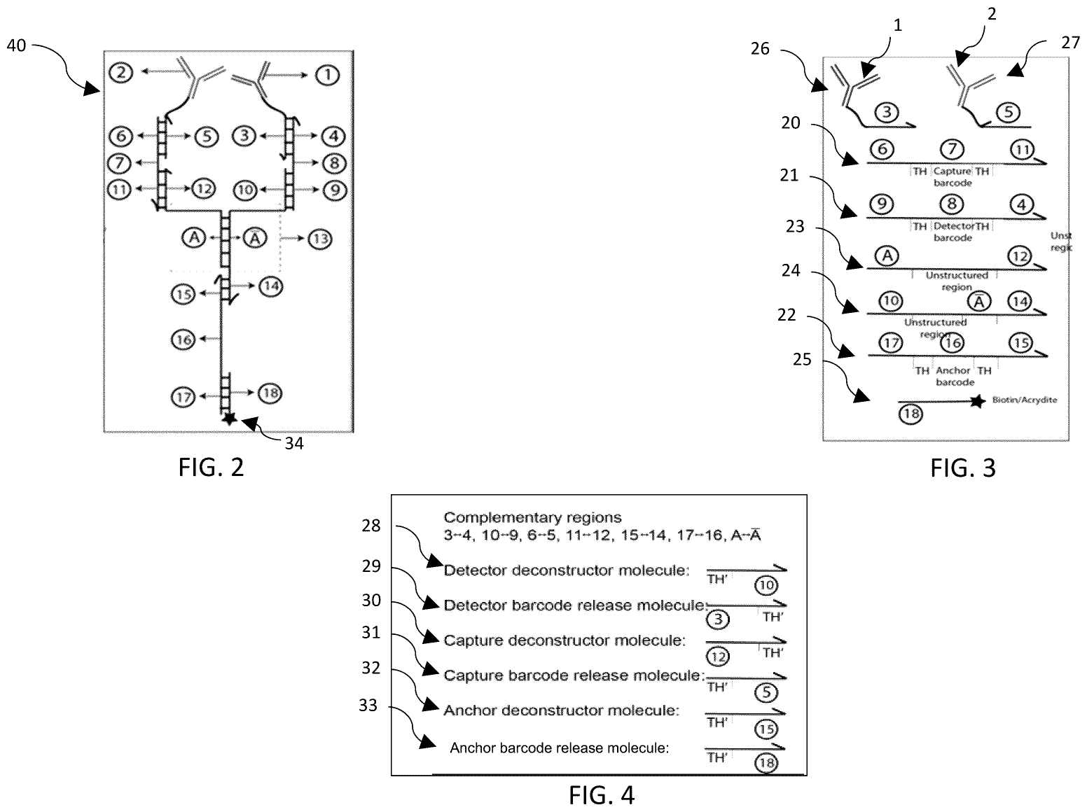

FIGS. 2-3 provides an exemplary depiction of a supramolecular structure 40 comprising a three arm nucleic acid junction and related subcomponents. FIG. 2 provides the complete supramolecular structure, while FIG. 3 provides the subcomponents that make up the supramolecular structure from FIG. 2. In some embodiments, the subcomponents of the supramolecular structure comprises five (5) DNA strands (ref. characters 20-24), one (1) DNA strand with a terminal modification 25, and two (2) antibodies (1,2) modified with a single DNA linker 3,5. FIG. 4 provides an exemplary depiction of the respective deconstructor molecules configured to cleave a respective subcomponent from the supramolecular structure 40 in FIG. 2. The references characters 1-18 in FIGS. 2-4 correspond to the respective components as provided with the same reference characters in FIG. 1.

As shown in FIGS. 2-3, in some embodiments of a supramolecular structure, the core structure comprises two strands, a first core strand 23 and a second core strand 24 that each comprise partially complementary DNA sequence domains labelled A and A respectively in the FIGS. 2-4.

In some embodiments, the first core strand 23 of the core structure comprises a first core linker 12 comprising a DNA sequence domain. In some embodiments, the first core strand 23 comprises the DNA sequence domain labelled as “A” in FIGS. 2-4, which is separated from the first core linker 12 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG.

In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the second core strand 24 of the core structure comprises a second core linker 10 comprising a DNA sequence domain. In some embodiments, the second core strand 24 comprises the DNA sequence domain labelled as “A” in FIGS. 2-4, which is separated from the second core linker 10 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG. In some embodiments, the second core strand 24 further comprises a third core linker 14 adjacent to the sequence domain “A”. In some embodiments, the third core linker 14 comprises a DNA sequence domain.

In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 17. In some embodiments, the unique anchor barcode sequence 16 comprises a nucleic acid (DNA or RNA) of a specific sequence. In some embodiments, the unique anchor barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators).

FIG. 4 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the second core strand 24. In some embodiments, the detector deconstructor molecule 28 is configured to cleave the link between the detector barcode 21 and the core structure (e.g., the second core strand 24). In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the first core strand 23. In some embodiments, a capture deconstructor molecule 30 is configured to cleave the link between the capture barcode and the core structure (e.g., the first core strand 23). In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the second core strand. In some embodiments, the anchor deconstructor molecule 32 is configured to cleave the link between the anchor barcode 22 and the core structure (e.g., the second core strand 24). In some embodiments, an anchor barcode release molecule 33 comprises a “TH′” domain, whose sequence is complementary to the “TH” domain on the anchor barcode 22 and the anchor molecule 18 (e.g., a DNA sequence domain). In some embodiments, the anchor barcode release molecule 33 is configured to cleave the link between the anchor barcode 22 and the anchor molecule 18.

In some embodiments, each of the different DNA domain sequences (reference character 1, 2, 3, 4, 5, 6, 7, 8, 9, 10, 11, 12, 13, 14, 15, A, Ā, TH, Capture Barcode 20, Detector Barcode 21 and Anchor barcode 22) independently comprise nucleic acid sequences from about 2 nucleotides to about 80 nucleotides.

### DNA Origami Based Supramolecular Structure

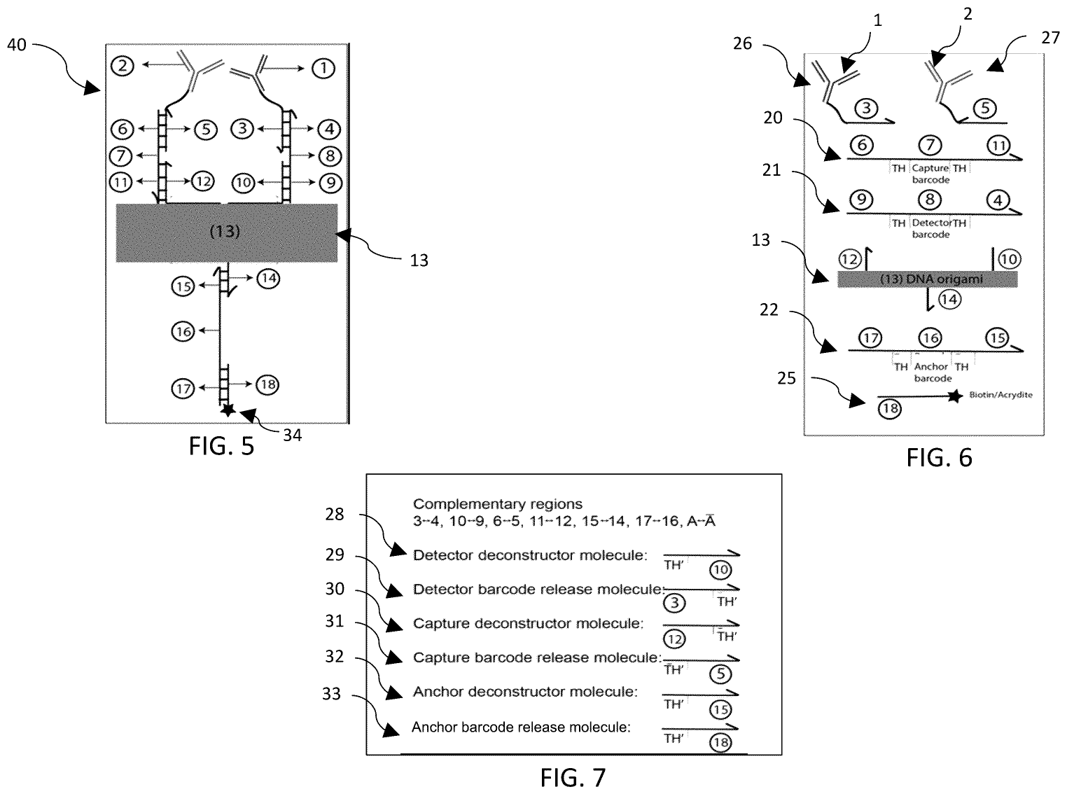

FIGS. 5-6 provides an exemplary depiction of a supramolecular structure 40 comprising a DNA origami and related subcomponents. FIG. 5 provides the complete supramolecular structure, while FIG. 6 provides the subcomponents that make up the supramolecular structure from FIG. 5. In some embodiments, the subcomponents of the supramolecular structure comprises a DNA origami 13 as a core structure, three (3) DNA strands (ref. characters 20-22), one (1) DNA strand with a terminal modification 25, and two (2) antibodies (1,2) modified with a single DNA linker 3,5. FIG. 6 provides an exemplary depiction of the respective deconstructor molecules configured to cleave a respective subcomponent from the supramolecular structure 40 in FIG. 5. The references characters 1-18 in FIGS. 5-7 correspond to the respective components as provided with the same reference characters in FIG. 1.

In some embodiments, the core structure 13 comprises a scaffolded DNA origami, wherein a circular ssDNA molecule, called “scaffold” strand, is folded into a predefined 2D or 3D shape by interacting with 2 or more short ssDNA, called “staple” strands, which interact with specific sub-sections of the ssDNA “scaffold” strand.

As shown in FIGS. 5-6, in some embodiments of a supramolecular structure, the core structure 13 comprises a DNA origami. In some embodiments, the core structure 13 comprises a first core linker 12 comprising a DNA sequence domain. In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the core structure 13 comprises a second core linker 10 comprising a DNA sequence domain. In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the unique detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the core structure 13 comprises a third core linker 14 that comprises a DNA sequence domain. In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 15, 17. In some embodiments, the unique detector barcode sequence 16 comprises a nucleic acid (DNA or RNA) of a specific sequence. In some embodiments, the unique detector barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators).

FIG. 6 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the core nanostructure 13. In some embodiments, the detector deconstructor molecule 28 is configured to cleave the link between the detector barcode 21 and the core structure 13. In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the core nanostructure 13. In some embodiments, a capture deconstructor molecule 30 is configured to cleave the link between the capture barcode 20 and the core structure 13. In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the core nanostructure 13. In some embodiments, the anchor deconstructor molecule 32 is configured to cleave the link between the anchor barcode 22 and the core structure 13. In some embodiments, an anchor barcode release molecule 33 comprises a TH′ domain, whose sequence is complementary to the TH domain on the anchor barcode 22 and the anchor molecule 18 (e.g., a DNA sequence domain) In some embodiments, the anchor barcode release molecule 33 is configured to cleave the link between the anchor barcode 22 and the anchor molecule 18.

### Stable and Unstable State of Supramolecular Structure

In some embodiments, the supramolecular structure comprises one or more stable state configurations. In some embodiments, the supramolecular structure comprises one or more unstable state configurations. In some embodiments, the supramolecular structure comprises a bi-stable configuration having a stable state configuration and an unstable state configuration. In some embodiments, the two states, stable and unstable are defined based on the ability of an individual supramolecular structure to remain structurally intact when subjected to a unique molecule (e.g., a deconstructor molecule) and/or a trigger signal. In some embodiments, when the supramolecular structure is in the stable state, then all the different components that are part of the supramolecular structure remain physically connected to each other even after being exposed to the deconstructor molecule and/or trigger signal. In some embodiments, when the supramolecular structure is in the unstable state, then the exposure to the deconstructor molecule and/or trigger signal leads to a defined section (e.g., one or more subcomponents) of the supramolecular structure being physically cleaved, i.e. unbound (separated) from the supramolecular structure. In some embodiments, the supramolecular structure is configured to shift from a stable state to an unstable state upon interaction with an analyte molecule (as described herein). In some embodiments, the supramolecular structure is configured to shift from a unstable state to a stable state upon interaction with an analyte molecule (as described herein). In some embodiment, the analyte molecule that triggers the state change of the supramolecular structure comprises a protein, clusters of proteins, peptide fragments, cluster of peptide fragments, DNA, RNA, DNA nanostructure, RNA nanostructures, lipids, an organic molecule, an inorganic molecule, or any combination thereof.

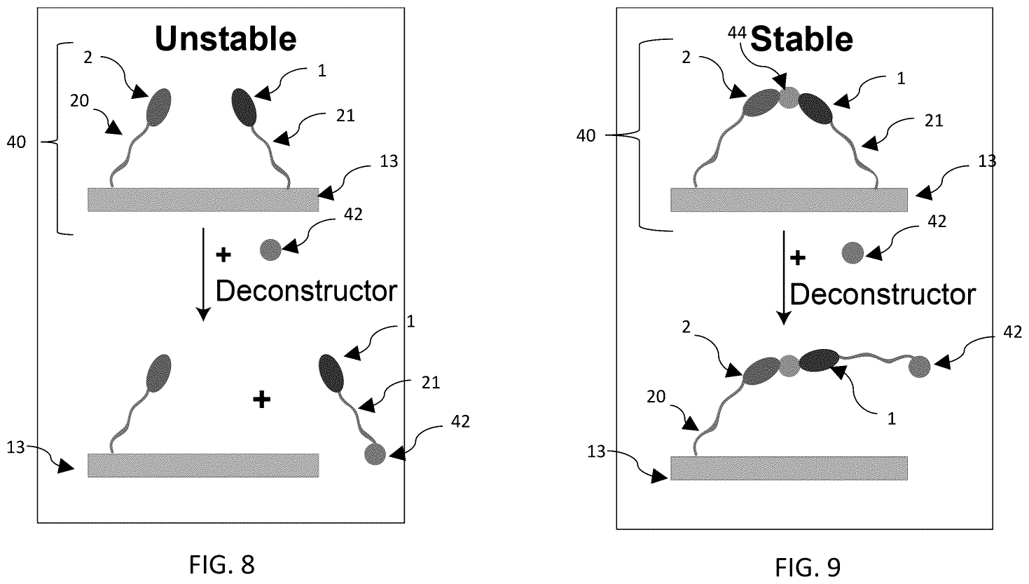

In some embodiments, a supramolecular structure in an unstable state configuration comprises a physical state wherein a linkage between the core structure 13 and a capture molecule 2 may be cleaved such that the capture molecule 2 is unbound from the core nanostructure 13. In some embodiments, the unstable state configuration comprises a physical state wherein a linkage between the core nanostructure 13 and a detector molecule 1 may be cleaved such that the detector molecule 1 is unbound from the core nanostructure 13. In some embodiments, the unstable state configuration comprises a physical state wherein a linkage between the core nanostructure 13 and a capture molecule 2 and a linkage between the core nanostructure 13 and a detector molecule 1 may be cleaved such that the capture molecule 2 and detector molecule 1 are unbound from the core nanostructure 13. In some embodiments, the linkage between the core nanostructure 13 and 1) the capture molecule 2, 2) the detector molecule 1, or 3) both, are cleaved upon being subjected to a trigger (e.g., a deconstructor molecule as described herein or trigger signal as described herein). FIG. 8 provides an exemplary depiction of a supramolecular structure 40 in an unstable state, wherein the detector molecule 1 is initially bound to the core structure 13 via a linkage with the detector barcode 21. With continued reference to FIG. 8, interaction with a deconstructor molecule 42 (e.g., detector deconstructor molecule 28) subsequently cleaves the linkage between the detector barcode 21 and core structure 13, such that the detector molecule 1 is unbound from the core nanostructure 13. In some embodiments, in the unstable state, the capture molecule 2 and detector molecule 1 on the core nanostructure 13 are freely diffusing with respect to each other, constrained only by the physical configuration of the core nanostructure 13.

In some embodiments, the stable state configuration comprises a physical state wherein the capture molecule 2 remains bound to the core nanostructure 13 upon cleavage of a linkage between the core structure 13 and the capture molecule 2. In some embodiments, the stable state configuration comprises a physical state wherein the detector molecule 1 remains bound to the core structure 13 upon cleavage of a linkage between the core nanostructure 13 and the detector molecule 1. In some embodiments, the stable state configuration comprises a physical state wherein the capture molecule 2 and detector molecule 1 are proximally positioned with respect to each other. In some embodiments, the detector molecule 1 and capture molecule 2 are proximally positioned with respect to each other with, or without, explicit bond formation between each other. In some embodiments, the detector 1 and capture 2 molecules are linked to each other. In some embodiments, the detector 1 and capture 2 molecules are linked to each other through a chemical bond. In some embodiments, the detector 1 and capture 2 molecules are linked together through a linkage with another molecule located between the capture and detector molecules (e.g., a sandwich formation). In some embodiments, the detector and capture molecules are linked together through linkage with an analyte molecule 44 from a sample (as described herein). FIG. 9 provides an exemplary depiction of a supramolecular structure 40 in a stable state, wherein the capture molecule 2 is linked to the detector molecule 1 through linkage with an analyte molecule 44. With continued reference to FIG. 9, interaction with a deconstructor molecule 42 cleaves the linkage between the detector molecule 1 and core structure 13, but the detector molecule 1 remains bound to the core nanostructure 13 through the linkage with the capture molecule 2. As described further herein, in some embodiments, a capture and/or detector molecule is configured to form a linkage with one or more specific types of analyte molecule from the sample. In some embodiments, interaction with the deconstructor molecule and/or trigger signal does not cleave the linkage between the capture and detector molecules.

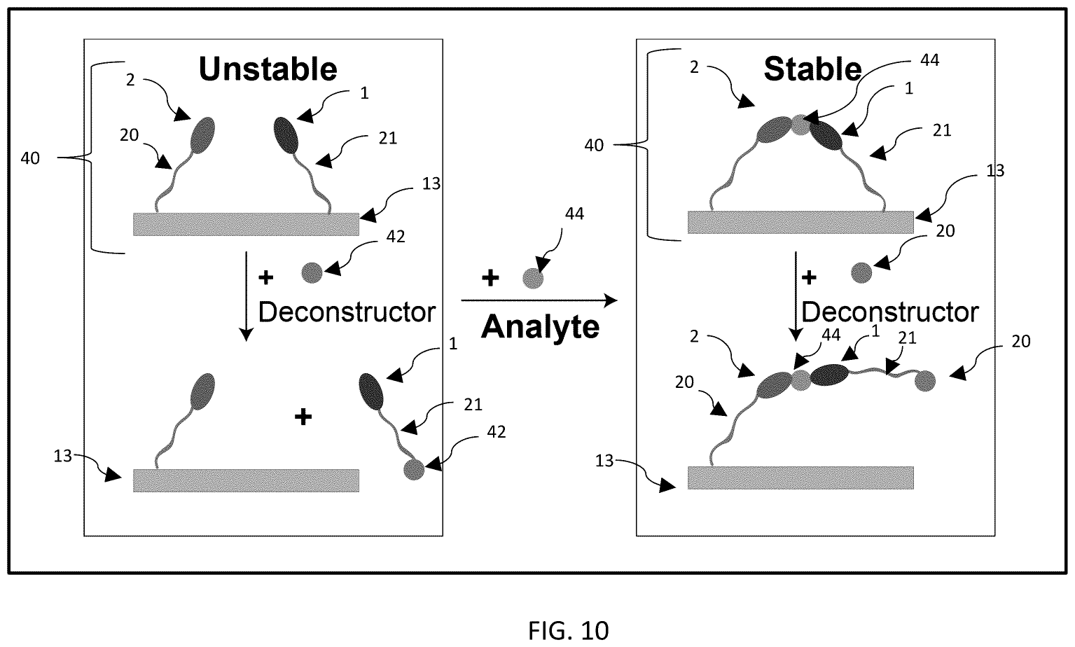

FIG. 10 provides an exemplary embodiment of a supramolecular structure shifting from an unstable state to a stable state. As described herein, a supramolecular structure 40 in an unstable state configuration will be separated from a detector molecule 1 (the detector molecule will be unbound from the supramolecular structure) upon interaction with a corresponding deconstructor molecule 42 (e.g., detector deconstructor molecule 28) and/or a trigger signal. With continued reference to FIG. 10, in some embodiments, interaction with an analyte molecule 44 from a sample binds the capture molecule and detector molecule together with the analyte molecule located therebetween (e.g., a sandwich formation), thereby shifting the supramolecular structure 40 from an unstable state to a stable state. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 10, with the supramolecular structure in a stable state, interaction with a corresponding deconstructor molecule cleaves the linkage between the core structure 13 and the detector barcode 21, wherein the detector molecule 1 remains linked to the core structure 13 through the linkage with the capture molecule 2 and analyte molecule 44.

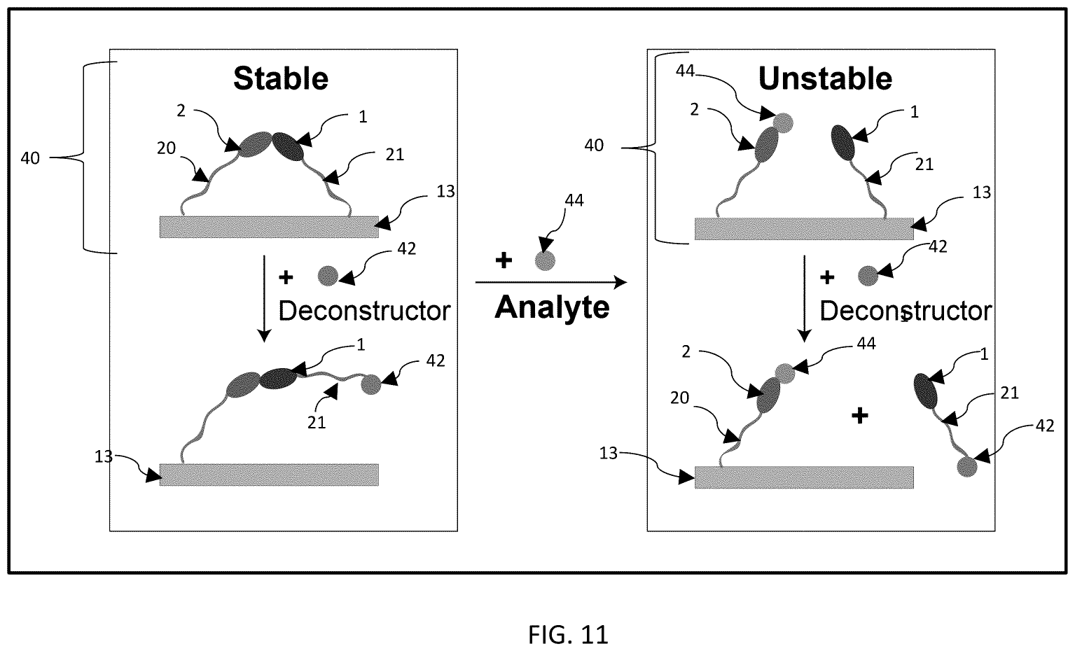

FIG. 11 provides an exemplary embodiment of a supramolecular structure 40 shifting from a stable state to an unstable state. As described herein, the supramolecular structure 40 is in a stable state configuration wherein the detector molecule 1 will remain linked to the core structure 13 upon interaction with a corresponding deconstructor molecule and/or trigger signal, due to the detector molecule 1 being linked to the capture molecule 2. With continued reference to FIG. 11, in some embodiments, interaction with an analyte molecule 44 from the sample cleaves the linkage between the capture molecule 2 and detector molecule 1, such that the analyte molecule 44 is bound to the capture molecule 1 only, thereby moving the supramolecular structure to an unstable state wherein the detector molecule 1 is bound to the core nanostructure 13 only through the linkage with the detector barcode 21. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 11, with the supramolecular structure in an unstable state, interaction with a corresponding deconstructor molecule 42 cleaves the linkage between the core structure 13 and the detector barcode 21, such that the detector molecule 1 is unbound (separated) from the core structure 13.

In some embodiments, the supramolecular structure 40 moves from a stable state to an unstable state upon interaction with an analyte molecule 44 that cleaves the linkage between a capture molecule 2 and detector molecule 1, wherein the analyte molecule 44 binds with the detector molecule 1. The capture molecule 2 is thereby unbound from the core structure 13 upon interaction with a corresponding destructor molecule 42 (e.g., capture deconstructor molecule 30).

### Methods for Detecting Analyte Molecules

As described herein, in some embodiments, one or more supramolecular structures enable the detection of one or more analyte molecules in a sample. In some embodiments, the supramolecular structure converts information about the presence of a given analyte molecule in a sample to a DNA signal. In some embodiments, the DNA signal corresponds to a capture barcode or detector barcode located on a supramolecular structure, wherein the capture molecule and detector molecule are simultaneously linked to the analyte molecule (e.g., sandwich formation). In some embodiments, capture and/or detector barcodes located on any unstable supramolecular structures are unbound therefrom using a trigger, such as a deconstructor molecule and/or a trigger signal. In some embodiments, the DNA signal is sequenced accordingly, and subsequently identified and correlated with the specific analyte molecule.

In some embodiments, detecting the presence of an analyte molecule, as described herein, comprises controllably releasing a single, or multiple, unique nucleic acid molecules into the solution to be used to identify as well as quantify properties of the analyte molecule from the sample that triggered the state change of the supramolecular structure. In some embodiments, said unique nucleic acid molecules are provided by capture barcodes and/or detector barcodes of the respective supramolecular structures. In some embodiments, detecting the presence of an analyte molecule, as described herein, comprises creating an optical or electrical signal connected to the state change that can be counted to quantify the concentration of the analyte molecule in solution. In some embodiments, the optical signal is generated by a DNA strand tagged with fluorescent labels. For example, in some embodiments, the optical signal comprises fluorescent emission. In some embodiments, the electrical signal is generated by a DNA strand with a signaling molecule, for instance methylene blue.

In some embodiments, a plurality of analyte molecules is simultaneously detected in a sample through multiplexing, wherein a plurality of supramolecular structures provides a plurality of signals (e.g., detector barcode, capture barcode) for sequencing and analyte identification. In some embodiments, methods described herein for detecting analytes in a sample provide a high-throughput and high-multiplexing capability by using a plurality of supramolecular structures. In some embodiments, the high-throughput and high-multiplexing capability provides high accuracy for analyte molecule detection and quantification. In some embodiments, methods described herein for detecting analytes in a sample are configured to characterize and/or identify biopolymers, including proteins molecules, quickly and at high sensitivity and reproducibility. In some embodiments, the plurality of supramolecular structures is configured to limit cross-reactivity associated errors. In some embodiments, such cross-reactivity associated errors comprise capture and/or detector molecules of a supramolecular structure interacting with capture and/or detector molecules of another supramolecular structure (e.g., intermolecular interactions). In some embodiments, each core structure of the plurality of supramolecular structures is identical to one another. In some embodiments, the structural, chemical, and physical property of each supramolecular structure is explicitly designed. In some embodiments, identical core structures have a prescribed shape, size, molecular weight, prescribed number of capture and detector molecules, predetermined distance between corresponding capture and detector molecules (as described herein), prescribed stoichiometry between corresponding capture and detector molecules, or combinations thereof, so as to limit the cross-reactivity between supramolecular structures. In some embodiments, the molecular weight of every core structure is identical and precise up to the purity of the core molecules. In some embodiments, each core structure has at least one capture molecule and at least one corresponding detector molecule.

In some embodiments, the plurality of supramolecular structures independently interacts with different analyte molecules from a sample since the state change (from unstable to stable) is driven primarily by intramolecular interaction (capture and detector molecules on the same supramolecular structure). In some embodiments, the plurality of supramolecular structures might share structural similarities due to certain subcomponents being the same, however the interaction between an analyte molecule from the sample and supramolecular structure is defined by the corresponding capture molecule and detector molecule. In some embodiments, each pair of detector and capture molecules on a given supramolecular structure may specifically interact with a particular analyte molecule in the sample, leading to a state change of supramolecular structure upon interacting with the particular analyte molecule. In some embodiments, each supramolecular structure comprises unique DNA barcodes corresponding to the respective pair of detector and capture molecules. In some embodiments, a pair of detector and capture molecules on a given supramolecular structure is designed to interact with more than one analyte molecule in the sample.

In some embodiments, each supramolecular structure is configured for single-molecule sensitivity to ensure the highest possible dynamic range needed to quantitatively capture the wide range of molecular concentrations within a typical complex biological sample. In some embodiments, single-molecule sensitivity comprises the capture and detector molecules of a given supramolecular structure configured to shift from an unstable state to a stable state (or vice versa) through binding with a single analyte molecule. In some embodiments, the plurality of supramolecular structures limit or eliminate the manipulation of the sample needed to reduce non-specific interaction as well as any user induced errors.

In some embodiments, the plurality of supramolecular structures is provided in a solution. In some embodiments, the plurality of supramolecular structures is attached to one or more substrates. In some embodiments, the plurality of supramolecular structures is attached to one or more widgets. In some embodiments, the plurality of supramolecular structures is attached to one or more solid substrates, one or more polymer matrices, one or more molecular condensates, or combinations thereof. In some embodiments, the one or more polymer matrices comprises one or more hydrogel particles. In some embodiments, the one or more polymer matrices comprises one or more hydrogel beads. In some embodiments, the one or more solid substrates comprises one or more planar substrates. In some embodiments, the one or more solid substrates comprises one or more microbeads. In some embodiments, the one or more solid substrates comprises one or more microparticles.

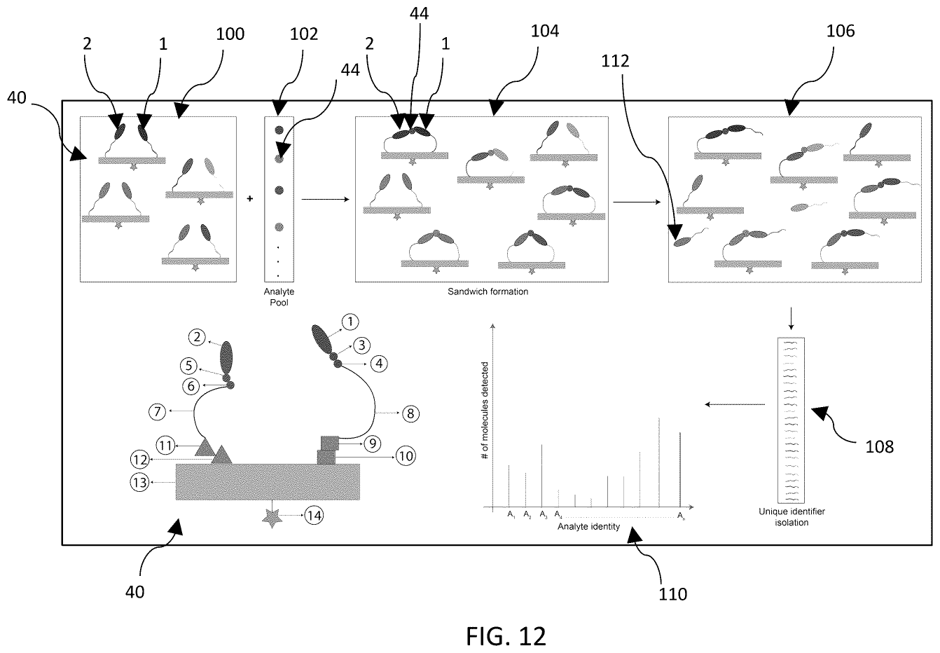

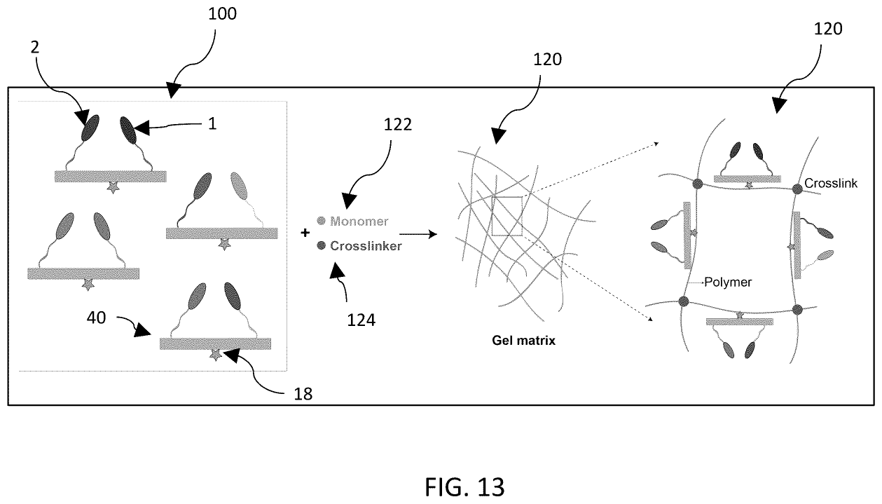

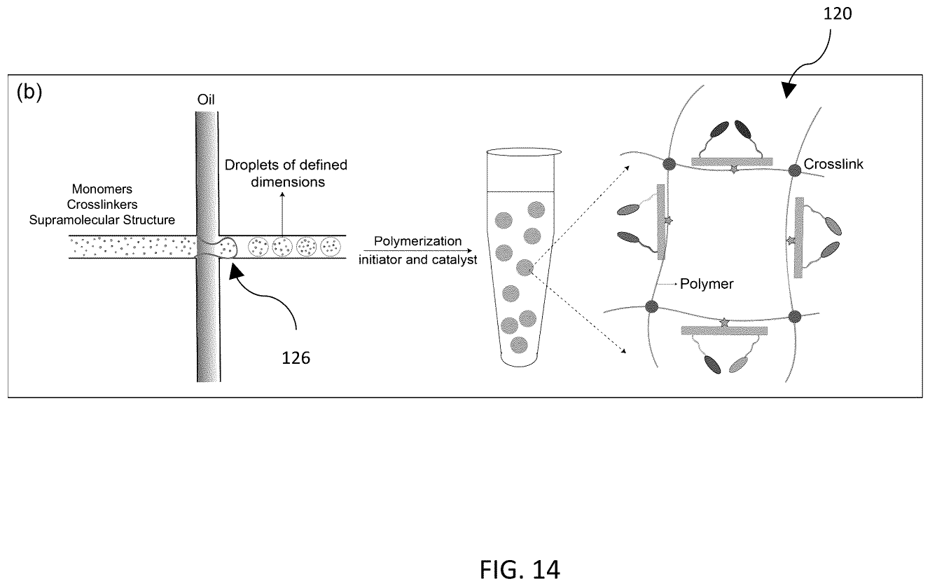

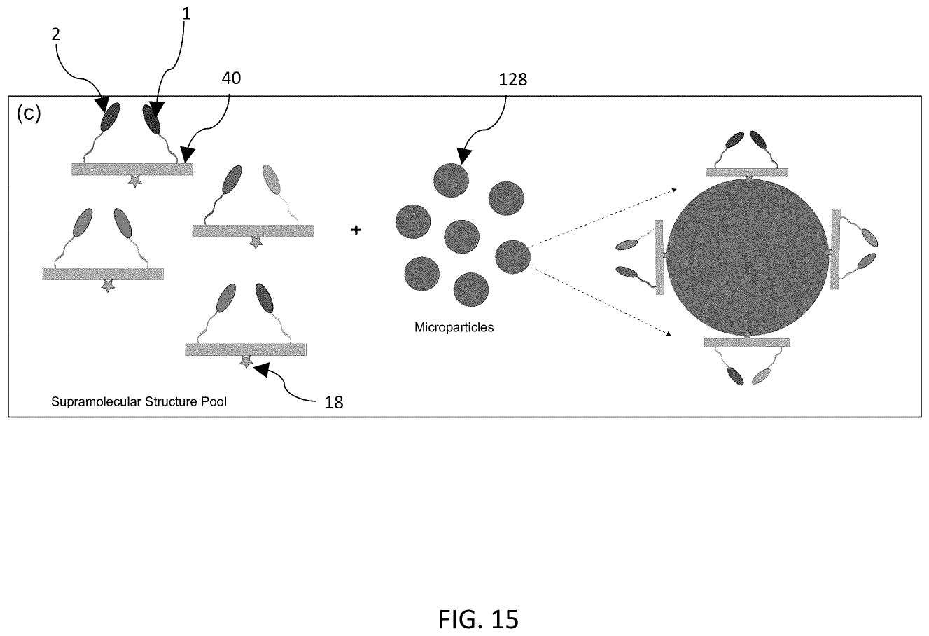

FIG. 12 provides an exemplary method for detecting one or more analyte molecules in a sample using one or more supramolecular structures. In some embodiments, the sample, comprising one or more analytes (e.g., analyte pool 102) is contacted with the one or more supramolecular structures 40 (e.g., supramolecular structure pool 100). In some embodiments, the supramolecular structures are attached to a plurality of widgets. In some embodiments, as described herein, the plurality of supramolecular structures is provided as being attached to one or more solid substrates, one or more polymer matrices, one or more molecular condensates, or combinations thereof. FIGS. 13-14 provide examples of supramolecular structures attached to a hydrogel bead (e.g., supramolecular structures embedded within a hydrogel bead). FIG. 15 provides an example of supramolecular structures attached to a solid substrate, e.g., a microparticle. In some embodiments, the sample comprises an aqueous solution, and is mixed with the supramolecular structures to form a combined solution. In some embodiments, contacting the sample with the supramolecular structures comprises incubating the sample with the supramolecular structures. In some embodiments, the sample and supramolecular structures are incubated in an incubator with prescribed environmental conditions. In some embodiments, the sample is incubated with the supramolecular structures for a time period from about 30 seconds to about 24 hours. In some embodiments, the sample is incubated with the supramolecular structures for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

With continued reference to FIG. 12, in some embodiments, the supramolecular structures are all in an unstable state (as shown with reference character 100). In some embodiments, and as described herein, interaction between an analyte molecule and corresponding capture 2 and detector 1 molecules shifts a respective supramolecular structure from an unstable state to a stable state (e.g., a sandwich formation with the capture molecule, analyte molecule, and detector molecule as shown with reference character 104). In some embodiments, a particular type of analyte molecule will bind with a particular pair of capture and detector molecules. In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the sample. In some embodiments, given that the state-change of the supramolecular structure is primarily dependent on the intra-molecular interactions (components located on the supramolecular structure), potential inter-molecular interactions between two different supramolecular structures are minimized or eliminated by limiting the net concentration of the supramolecular structures in the combined solution, such that the mean distance between any two supramolecular structures is larger than maximum intramolecular distance between a pair of capture and detector molecules on a given supramolecular structure.

As seen in FIG. 12, reference character 104, after contacting the sample, at least one of the supramolecular structures shifted to a stable state through interaction with an analyte molecule (e.g., sandwich formation wherein the capture molecule, analyte molecule and detector molecule are simultaneously linked together), while at least one of the supramolecular structures remained in an unstable state as the respective capture and detector molecules did not bind or interact with an analyte molecule from the sample.

After the sample has been contacted with the supramolecular structures for a prescribed amount of time, the combined solution of the sample and supramolecular structures, as shown in FIG. 12, is subjected to a trigger so as to cleave a linkage between the detector molecule and the core structure (reference character 106). In some embodiments, the trigger comprises introducing a solution comprising one or more deconstructor molecules (e.g., detector deconstructor molecule, reference character 28 from FIGS. 4,7) to the combined solution. In some embodiments, the trigger comprises subjecting the combined solution to a trigger signal. In some embodiments, the trigger comprises a combination of introducing a deconstructor molecule in the combined solution and subjecting the combined solution to a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the combined solution is subjected to the trigger for a prescribed amount of time. In some embodiments, the combined solution is incubated with one or more deconstructor molecules for a prescribed amount of time. In some embodiments, the combined solution is incubated with the deconstructor molecules for a time period from about 30 seconds to about 24 hours. In some embodiments, the combined solution is incubated with the deconstructor molecules for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

As shown in FIG. 12 reference character 106, in some embodiments, subjecting the combined solution to the trigger cleaves a linkage between the detector molecule and core structure of a supramolecular structure, such as the linkage between a detector barcode (e.g., reference character 21 from FIG. 1) and the core structure 13. In some embodiments, the cleavage is achieved through nucleic acid (DNA/RNA) strand displacement, optical cleavage, chemical cleavage, another technique known in the art, or combinations thereof. For supramolecular structures that shifted to a stable state, the detector molecule 1 is shown as remaining to be linked to the core structure 13 via linkage with the corresponding capture molecule 2. For supramolecular structures that remained in an unstable state, the detector molecule is shown as being unbound 112 from the respective supramolecular structure. In some embodiments, the unbound detector molecules 1 remain linked to the respective detector barcodes 21.

As used herein, the term “strand displacement” refers to a molecular tool to exchange one strand of DNA or RNA (output) with another strand (input). It is based on the hybridization of two complementary strands of DNA or RNA. It starts with a double-stranded DNA complex composed of the original strand and the protector strand. The original strand has an overhanging region the so-called “toehold” (TH) which is complementary to a third strand of DNA referred to as the “invading strand”. Accordingly, for example, the invading strand is a sequence of single-stranded DNA (ssDNA) which is complementary to the original strand. The toehold regions initiate the process by allowing the complementary invading strand to hybridize with the original strand, creating a DNA complex composed of three strands of DNA. After the binding of the invading strand and the original strand occurred, branch migration of the invading domain then allows the displacement of the initial hybridized strand.

In some embodiments, the unbound detector molecules 1 (and corresponding detector barcode 21) are further separated from the combined solution. In some embodiments, the unbound detector molecules are separated from the combined solution through polyethylene glycol (PEG) precipitation. In some embodiments, the unbound detector molecules are separated from the combined solution by binding each core structure in the combined solution to microbeads, a solid support and/or magnetic beads through a corresponding anchor molecule on the respective core structure, followed by separation of the unbound detector molecules through centrifugation, micron filtration, chromatography or combinations thereof.

In some embodiments, after the unbound detector molecules have been separated from the combined solution, the detector barcodes 21 are cleaved from the corresponding detector molecules that are linked to a respective capture molecule (e.g., as located on a supramolecular structure that shifted to a stable state). In some embodiments, the detector barcodes 21 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a detector barcode release molecule (e.g., reference character 29 from FIGS. 4 and 7).

In some embodiments, the cleaved detector barcodes 21 are isolated (reference character 108 FIG. 12) from the solution comprising the supramolecular structures. In some embodiments, the cleaved detector barcodes 21 are isolated from the solution through polyethylene glycol (PEG) precipitation. In some embodiments, the cleaved detector barcodes 21 are isolated from the solution by binding the core structures in the solution to microbeads, solid support and/or magnetic beads through a corresponding anchor molecule on the respective core structure, followed by isolation of the cleaved detector barcodes through centrifugation, micron filtration, chromatography or combinations thereof.

In some embodiments, the cleaved detector barcodes provide a signal that correlates to the respective analyte molecule bound to the respective detector molecule. In some embodiments, as described herein, the detector barcode comprises a DNA strand. In some embodiments, the detector barcode provides a DNA signal correlating to the analyte molecule. In some embodiments, as depicted in FIG. 12 reference character 110, the isolated detector barcodes 21 are analyzed to identify and/or quantify the corresponding analyte molecules in the sample. In some embodiments the analysis of the isolated detector barcodes comprises genotyping, qPCR, sequencing, or combinations thereof.

In some embodiments, the method for detecting analyte molecules as depicted in FIG. 12 comprises cleaving the capture barcode 20 from a corresponding capture molecules that are linked to a respective detector molecule (e.g., as located on a supramolecular structure that shifted to a stable state). In some embodiments, the capture barcodes 20 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a capture barcode release molecule (e.g., reference character 31 from FIGS. 4 and 7).

In some embodiments, the cleaved capture barcodes 20 are isolated (reference character 108 FIG. 12) from the solution comprising the supramolecular structures. In some embodiments, the cleaved capture barcodes 20 are isolated from the solution through polyethylene glycol (PEG) precipitation. In some embodiments, the cleaved capture barcodes 20 are isolated from the solution by binding the core structures in the solution to microbeads, solid support and/or magnetic beads through a corresponding anchor molecule on the respective core structure, followed by isolation of the cleaved capture barcodes through centrifugation, micron filtration, chromatography or combinations thereof.

In some embodiments, the cleaved capture barcodes provide a signal that correlates to the respective analyte molecule bound to the respective detector molecule. In some embodiments, as described herein, the capture barcode comprises a DNA strand. In some embodiments, the capture barcode provides a DNA signal correlating to the analyte molecule. In some embodiments, as depicted in FIG. 12 reference character 110, the isolated capture barcodes 21 are analyzed to identify and/or quantify the corresponding analyte molecules in the sample. In some embodiments the analysis of the isolated capture barcodes comprises genotyping, qPCR, sequencing, or combinations thereof.

Supramolecular Structures Provided with Hydrogel Beads or Solid Substrate

As described herein, in some embodiments, one or more supramolecular structures are provided with one or more hydrogel beads and/or one or more solid substrates. In some embodiments, the hydrogel bead comprises one or more supramolecular structures polymerized to a hydrogel matrix. FIG. 13 provides an exemplary embodiment for forming a hydrogel bead 120, wherein in addition to combining one or more monomers 122 and one or more crosslinking molecules 124 to form a hydrogel, one or more supramolecular structures 40 are introduced. In some embodiments, the one or more supramolecular structures 40 co-polymerizes with the hydrogel matrix, forming the hydrogel bead 120. In some embodiments, hydrogel bead 120 comprises the one or more supramolecular structures attached to the hydrogel matrix. In some embodiments, the hydrogel bead 120 comprises the one or more supramolecular structures embedded within the hydrogel matrix. In some embodiments, each respective anchor molecule 18 of the one or more supramolecular structures 40 co-polymerizes with the hydrogel matrix 120. In some embodiments, the one or more monomers 122 comprise an acrylamide. In some embodiments, the one or more cross-linkers 124 comprise a bis-acrylamide. In some embodiments, each hydrogel bead is formed using microfabrication tools. In some embodiments, each hydrogel bead is formed using emulsion polymerization. FIG. 14 provides an exemplary embodiment for forming a hydrogel bead 120, which comprises trapping 126 one or more monomers, one or more crosslinkers, and one or more supramolecular structures 40 within a droplet. In some embodiments, the droplet is an oil droplet. In some embodiments, the droplet dimension are specified. In some embodiments, polymerization occurs within the droplet thereby forming the one or more hydrogel beads 120. In some embodiments, polymerization occurs through interaction with an initiator and/or catalyst.

FIG. 15 provides an exemplary embodiment wherein one or more supramolecular structures 40 are attached to a solid substrate 128. In some embodiments, each anchor molecule 18 of a supramolecular structure 40 links with the solid surface of the solid substrate 128. In some embodiments, the solid substrate 128 comprises a microparticle. In some embodiments, the microparticle comprises a polystyrene particle, silica particle, magnetic particle or paramagnetic particle. In some embodiments, the solid substrate 128 comprises a microbead. In some embodiments, the microbead comprises a polystyrene bead, silica bead, magnetic bead or paramagnetic bead.

As described herein, in some embodiments, a plurality of supramolecular structures embedded within a single hydrogel bead or attached to a solid substrate are spaced apart with a prescribed distance so as to limit or eliminate cross-reactivity (cross-talk, intermolecular interaction) with other supramolecular structures. In some embodiments, the number, size, and/or stoichiometry of the supramolecular structures attached to each hydrogel bead or solid substrate are specified so as to achieve a prescribed distance between each supramolecular structure. In some embodiments, the surface and volumetric density of the supramolecular structures attached to each hydrogel bead or solid substrate are controlled to minimize or eliminate intermolecular interactions and thereby reducing the possibility of cross-talk between the plurality of supramolecular structures. In some embodiments, the distance between any two supramolecular structures on a given hydrogel bead or solid substrate (e.g., microparticle) is larger than a maximum distance between capture and detector molecules of a supramolecular structure, so as to minimize intermolecular interactions between molecules from different supramolecular structures.

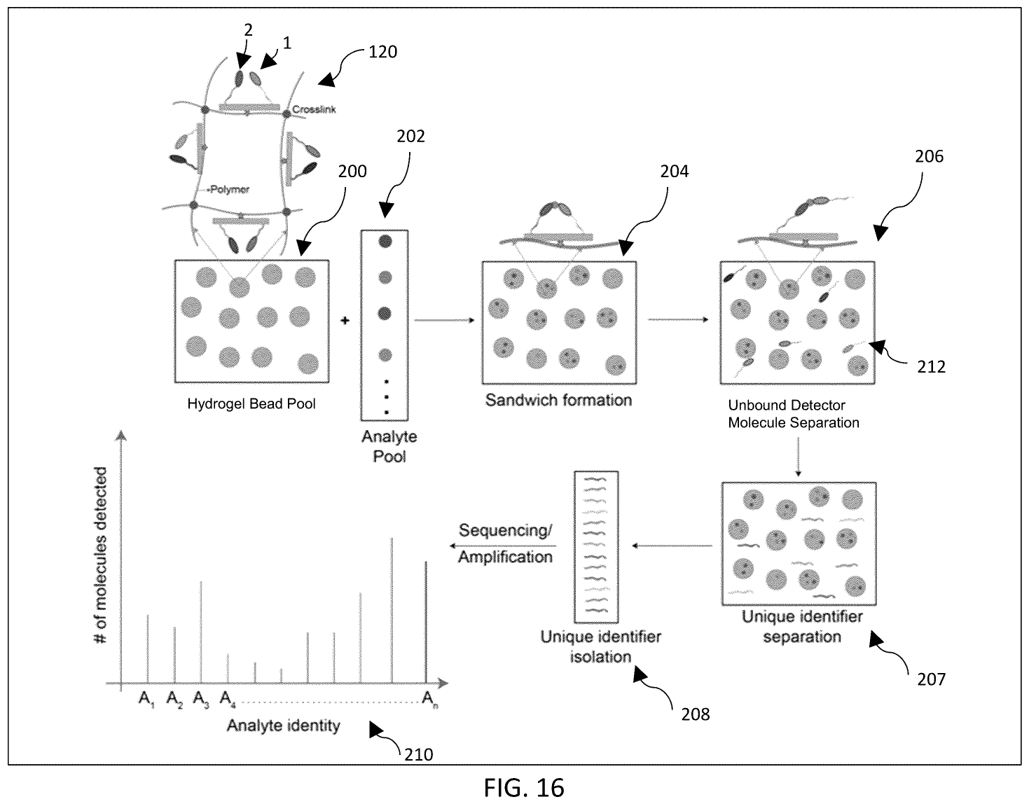

FIG. 16 provides an exemplary method for detecting one or more analyte molecules in a sample using one or more supramolecular structures embedded within one or more hydrogel beads or attached to one or more solid substrates (e.g., microparticles). FIG. 16 depicts an exemplary embodiment where a hydrogel bead pool 200 is provided, wherein one or more supramolecular structures are embedded within one or more hydrogel beads 120. In some embodiments, alternate to a hydrogel bead pool, a solid substrate pool is provided, wherein one or more supramolecular structures are attached to one or more solid substrates (e.g., microparticle), as described herein and shown in FIG. 15. In some embodiments, the sample, comprising one or more analyte molecules (e.g., analyte pool 202) is contacted with the supramolecular structures embedded within the hydrogel beads 120. In some embodiments, the sample comprises an aqueous solution, and is mixed with the hydrogel bead pool 200 to form a combined solution. In some embodiments, contacting the sample with the supramolecular structures comprises incubating the sample with the supramolecular structures. In some embodiments, the sample and supramolecular structures are incubated in an incubator with prescribed environmental conditions. In some embodiments, the sample is incubated with the supramolecular structures for a time period from about 30 seconds to about 24 hours. In some embodiments, the sample is incubated with the supramolecular structures for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

With continued reference to FIG. 16, in some embodiments, the supramolecular structures are all in an unstable state (as shown with the exemplary hydrogel bead 120). In some embodiments, and as described herein, interaction between an analyte molecule and corresponding capture 2 and detector 1 molecules shift the respective supramolecular structure from the unstable state to a stable state (e.g., a sandwich formation with the capture molecule, analyte molecule, and detector molecule as shown with reference character 204). In some embodiments, a particular type of analyte molecule will bind with a particular pair of capture and detector molecules. In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the sample. In some embodiments, given that the state-change of the supramolecular structure is primarily dependent on the intra-molecular interactions (on the supramolecular nanostructure), potential intermolecular interactions between two different supramolecular structures are minimized or eliminated by limiting the net concentration of the supramolecular structures embedded within a hydrogel bead or attached to a solid substrate (as described herein), such that the mean distance between any two supramolecular structures is larger than maximum intramolecular distance between a pair of capture and detector molecules on a given supramolecular structure.

In some embodiments, after contacting the sample, at least one of the supramolecular structures moves to a stable state (e.g., sandwich formation wherein the capture molecule, analyte molecule and detector molecule are simultaneously linked together), while at least one of the supramolecular structures remains in an unstable state as the respective capture and detector molecules did not bind or interact with an analyte molecule from the sample.

With continued reference to FIG. 16, after the sample has been contacted with the supramolecular structures for a prescribed amount of time, the combined solution of the sample and supramolecular structures is subjected to a trigger so as to cleave a linkage between the detector molecule and the core structure for the respective supramolecular structures. In some embodiments, the trigger comprises introducing a solution comprising one or more deconstructor molecules (e.g., detector deconstructor molecule, reference character 28 from FIGS. 4,7) to the combined solution. In some embodiments, the trigger comprises subjecting the combined solution to a trigger signal. In some embodiments, the trigger comprises a combination of introducing a deconstructor molecule in the combined solution and subjecting the combined solution to a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the combined solution is subjected to the trigger for a prescribed amount of time. In some embodiments, the combined solution is incubated with one or more deconstructor molecules for a prescribed amount of time. In some embodiments, the combined solution is incubated with the deconstructor molecules for a time period from about 30 seconds to about 24 hours. In some embodiments, the combined solution is incubated with the deconstructor molecules for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

As shown in FIG. 16, reference character 206, in some embodiments, subjecting the combined solution to the trigger cleaves a linkage between a detector molecule and respective core structure, such as through the linkage between a detector barcode (e.g., reference character 21 FIG. 1) and the core structure 13. In some embodiments, the cleavage is achieved through nucleic acid (DNA/RNA) strand displacement, optical cleavage, chemical cleavage, another technique known in the art, or combinations thereof. For supramolecular structures that moved to a stable state, the detector molecule 1 is shown as remaining to be linked to the core structure 13 via linkage with the corresponding capture molecule 2. For supramolecular structures that remained in an unstable state, the detector molecule is shown as being unbound 212 from the respective core structure. In some embodiments, the unbound detector molecules are separated from the hydrogel bead (or solid substrate), as shown with reference character 206. In some embodiments, the unbound detector molecules 2 remain linked to the respective detector barcodes 21.

In some embodiments, the unbound detector molecules and corresponding detector barcodes are further separated from the combined solution. In some embodiments, the unbound detector molecules are separated from the combined solution through polyethylene glycol (PEG) precipitation. In some embodiments, the unbound detector molecules are separated from the combined solution by binding the core structures in the combined solution to microbeads, solid support and/or magnetic beads through a corresponding anchor molecule on the respective core structure, followed by separation of the unbound detector molecules through centrifugation, micron filtration, chromatography or combinations thereof.

In some embodiments, after the unbound detector molecules have been separated from the combined solution, the detector barcodes 21 are cleaved from the corresponding detector molecules linked to a respective capture molecule (e.g., as located on a supramolecular structure that shifted to a stable state). In some embodiments, the detector barcodes are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a detector barcode release molecule (e.g., reference character 29 from FIGS. 4 and 7).

As shown with reference character 207 in FIG. 16, in some embodiments, the cleaved detector barcodes 21 are separated from the corresponding hydrogel bead or solid substrate. In some embodiments, the cleaved detector barcodes 21 are isolated (reference character 208 FIG. 16) from the solution comprising the supramolecular structures. In some embodiments, the cleaved detector barcodes 21 are isolated from the solution through polyethylene glycol (PEG) precipitation. In some embodiments, the cleaved detector barcodes 21 are isolated from the solution by binding the core structures in the solution to microbeads, solid support and/or magnetic beads through a corresponding anchor molecule on the respective core structure, followed by isolation of the cleaved detector barcodes through centrifugation, micron filtration, chromatography or combinations thereof.

In some embodiments, the cleaved detector barcodes 21 provide a signal that correlates to the analyte molecule bound to the respective detector molecule. In some embodiments, as described herein, the detector barcode comprises a DNA strand. In some embodiments, the detector barcode provides the DNA signal correlating to the analyte molecule. In some embodiments, as depicted in FIG. 16 reference character 210, the isolated detector barcodes 21 are analyzed to identify the corresponding analyte in the sample. In some embodiments, the isolated detector barcodes 21 are analyzed to identify and/or quantify the corresponding analyte molecules in the sample. In some embodiments the analysis of the isolated detector barcodes comprises genotyping, qPCR, sequencing, or combinations thereof.

Detecting Analyte Molecules within a Single Cell

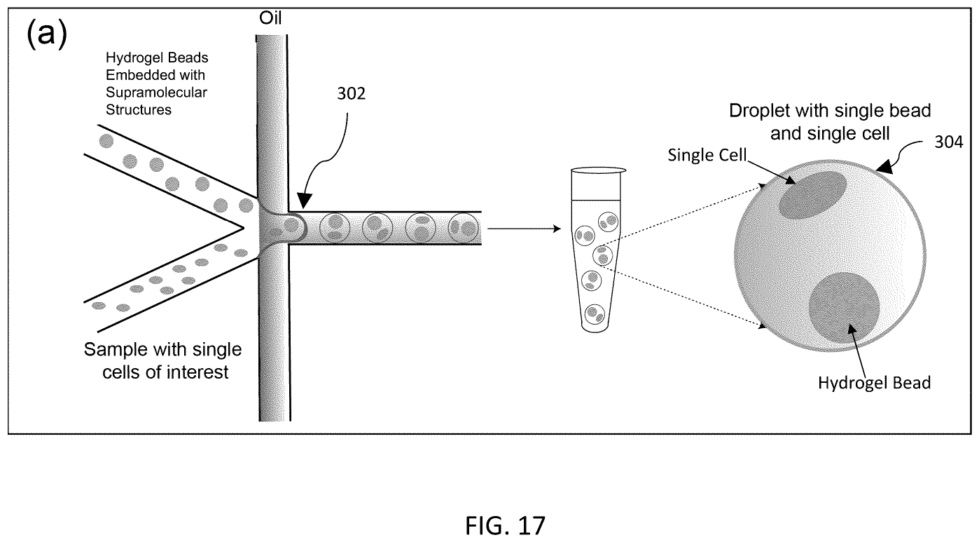

FIGS. 17-20 provide an exemplary method for detecting analyte molecules located within a single cell. In some embodiments, attaching supramolecular structures to a hydrogel bead, or attaching supramolecular structures onto a solid substrate (e.g., microbeads), enables the detection of intracellular analyte molecules (e.g., protein, antigen) and quantification of intracellular analyte molecules (e.g., protein, antigen) at single cell resolution. In some embodiments, the intracellular analyte molecules may not exist outside the respective cell. In some embodiments, the detection of intracellular analyte molecules (e.g., protein, antigen) and quantification of intracellular analyte molecules (e.g., protein, antigen) at single cell resolution comprises a single-cell proteomics assay. FIGS. 17-20 provide an exemplary method for detecting analyte molecules wherein the supramolecular structures are provided as embedded within hydrogel beads. In some embodiments, the method depicted in FIGS. 17-20 alternatively comprises providing the supramolecular structures as attached to solid substrates (e.g., microbeads).

FIG. 17 provides an exemplary first step comprising using a microfluidic droplet formation chip to trap 302 single cells with hydrogel beads that are attached with one or more supramolecular structures, wherein each droplet 304 formed encloses a single cell and hydrogel bead. In some embodiments, each droplet 304 encloses one or more single cells and one or more hydrogel beads. In some embodiments, the supramolecular structures are attached to the hydrogel beads (e.g., embedded within the hydrogel beads) using methods as described herein (e.g., FIGS. 13-14). In some embodiments, the one or more supramolecular structures are configured to interact with specific intercellular analyte molecules (e.g., proteins, antigens). In some embodiments, other methods and/or microfluidic chip designs are used to achieve the trapping of single cells with the one or more hydrogel beads.

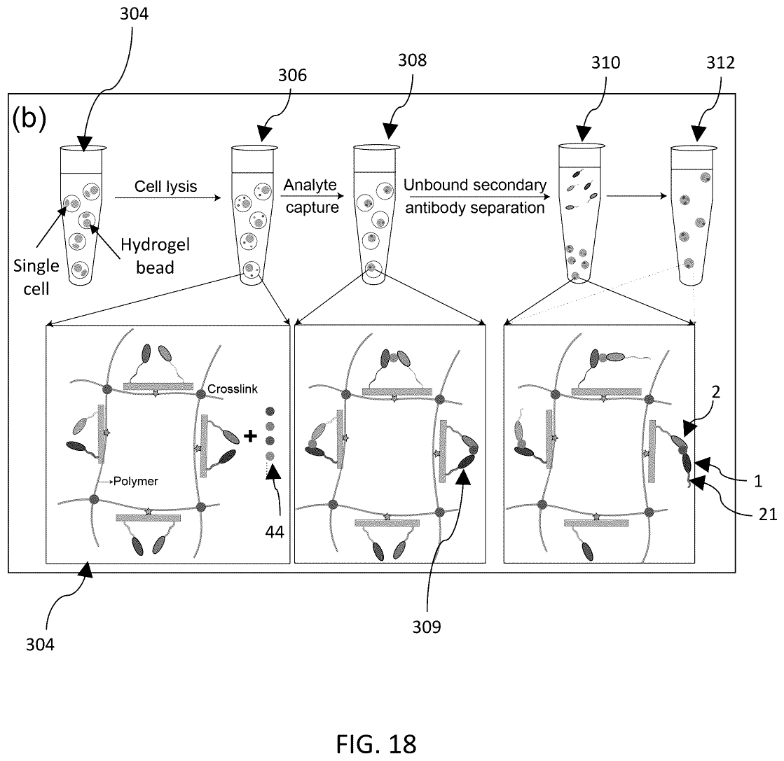

FIG. 18 provides an exemplary embodiment for collecting the droplets 304, having the trapped single cells and hydrogel beads, in a combined solution, and processing the droplets 304. In some embodiments, the intracellular analyte molecules (e.g., proteins, antigens) are transferred from each cell onto or about the hydrogel bead within the same droplet 304. In some embodiments, transferring the intracellular analyte molecules comprises lysing the cell that is trapped in the droplet (reference character 306 in FIG. 18, step 1). In some embodiments, lysing comprises mechanical processing or introducing a lysis buffer. As depicted in the combined solution, in some embodiments, all the supramolecular structures corresponding to a hydrogel bead is in an unstable state. In some embodiments, the contents of the lysate (e.g., analyte molecules 44) is subsequently allowed to interact 308 (step 2) with the hydrogel bead within the droplet, thereby enabling specific intercellular analyte molecules (e.g., proteins, antigens) to be captured by associated supramolecular structures attached with the hydrogel bead (e.g., capture 2 and detector 1 molecules). In some embodiments, the contents of the lysate (e.g., analyte molecules) is allowed to interact with the hydrogel bead for a time period from about 30 seconds to about 24 hours. In some embodiments, the contents of the lysate (e.g., analyte molecules) is allowed to interact with the hydrogel bead for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

In some embodiments, and as described herein, interaction between an analyte molecule and corresponding capture 2 and detector 1 molecules shifts the respective supramolecular structure from the unstable state to a stable state (as shown with reference character 309). In some embodiments, a particular type of analyte molecule will bind with a particular pair of capture and detector molecules (e.g., a sandwich formation with the capture molecule, analyte molecule, and detector molecule). In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the cell.

After the contents of the lysate have been allowed to interact with the hydrogel for a prescribed amount of time, in some embodiments, the droplets are subsequently broken, after which the hydrogel beads are washed. In some embodiments, the hydrogel beads are subjected to a trigger so as to cleave a linkage between the detector molecule and the core structure for the respective supramolecular structures. In some embodiments, the trigger comprises introducing a solution comprising one or more deconstructor molecules (e.g., detector deconstructor molecule 28 from FIGS. 4,7) to the combined solution comprising the hydrogel beads (reference character 310). In some embodiments, the trigger comprises subjecting the combined solution to a trigger signal. In some embodiments, the trigger comprises subjecting the combined solution to a deconstructor molecule and a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the hydrogel beads are subjected to the trigger for a prescribed amount of time. In some embodiments, the hydrogel beads are subjected to the trigger from about 30 seconds to about 24 hours. In some embodiments, the hydrogel beads are subjected to the trigger from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

In some embodiments, the trigger (e.g., detector deconstructor molecule 28 from FIGS. 4,7) releases all the detector molecules from the hydrogel beads that are not linked to a corresponding capture molecule (i.e. not participating in the sandwich formation comprising a capture molecule, detector molecule, and analyte molecule).

In some embodiments, after the hydrogel beads have been subjected to the trigger for a prescribed amount of time, the hydrogel beads are washed one or more times to remove any weakly bound analyte molecules (e.g., proteins, antigens) or any detector molecules unbound from a respective supramolecular structure (reference character 312, step 4). In some embodiments, after being washed a prescribed number of times, each hydrogel bead contains analyte molecules (e.g., protein, antigens) that have been specifically captured from a single cell.

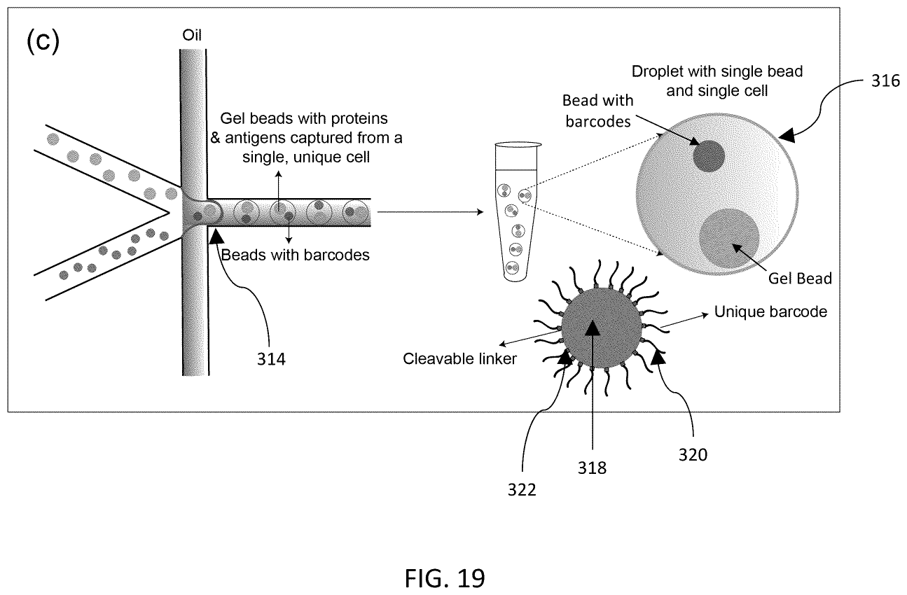

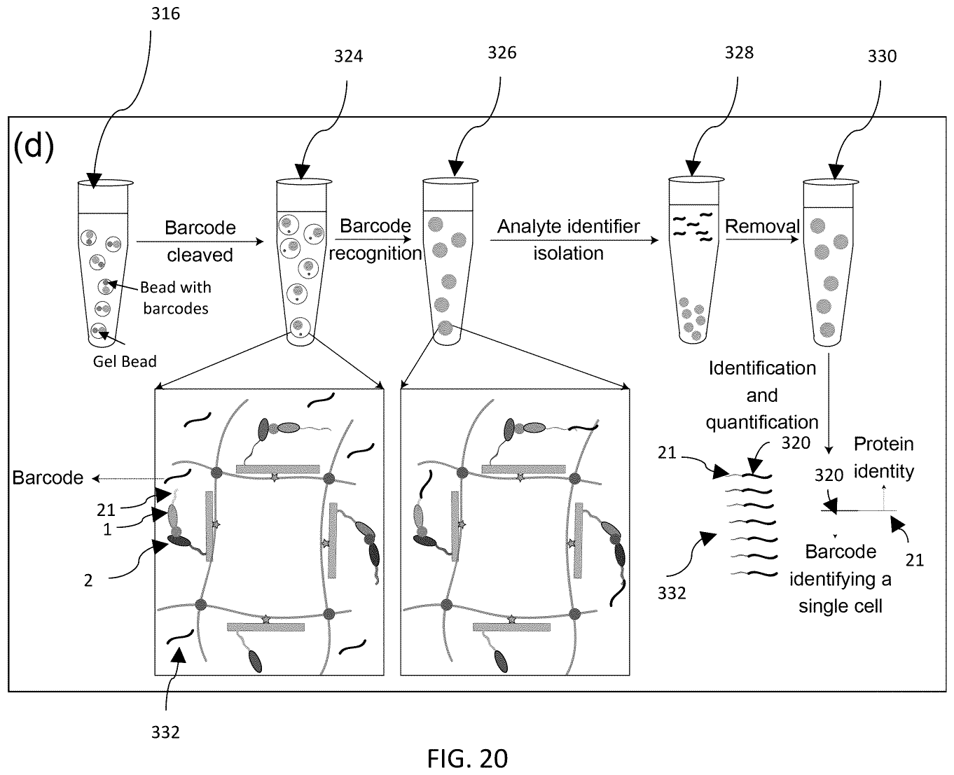

FIGS. 19-20 provide an exemplary depiction of a method for analyzing the content of each resulting hydrogel bead from the method depicted in FIG. 18. In some embodiments, the content of each hydrogel bead is analyzed independently, wherein each hydrogel bead is barcoded individually. FIG. 19 provides an exemplary illustration of a microfluidic droplet formation system that is designed to form droplets 316 that enclose 314 1) a single hydrogel bead, that is carrying within itself one or more analyte molecules (e.g., protein, antigen) from a single cell, with 2) a unique barcode bead 318. In some embodiments, each barcode bead comprises a unique nucleic acid strand 320 that is between 20 and 60 bases long and connected to the bead through a linker 322 that can be cleaved. In some embodiments, the cleavable linker on the barcode bead is broken using an electromagnetic signal (light) or a chemical signal.

FIG. 20 provides an exemplary illustration of a method for transferring the unique barcode onto each hydrogel bead, both of which are present in a single droplet 316 (reference character 324, step 1). In some embodiments, the barcode 320 is cleaved from the barcoding beads 318 and allowed to interact with the hydrogel beads in the respective droplet 316. As described herein, the barcode 320 is cleaved from the barcoding bead 318 by being subjected to an electromagnetic signal (e.g., light, UV light, DTT) or chemical signal. In some embodiments, cleaving the barcode 320 from the barcoding bead leads to the barcode 320 binding to a detector barcode 21 on a supramolecular structure within the respective droplet 316 (reference character 326, step 2). In some embodiments, the droplets are subsequently broken. In some embodiments, barcode strands 320 that did not bind with a detector barcode are separated (reference character 328). In some embodiments, the hydrogel beads are washed to remove any remaining barcoding strands from the solution (reference character 320). In some embodiments, the barcoded detector barcodes 332 are separated from the detector molecules and further analyzed. In some embodiments, the barcoded detector barcodes 332 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof.

In some embodiments, each separated barcoded detector barcode 332 will have two sections: a first section 320 that has the unique barcode 320 that identifies a unique cell, and a second section 21 that provides the identity of the analyte molecule (e.g., protein or antigen). In some embodiments, taken together, the analysis of the barcoded detector barcode 332 enables the concentration of intracellular analyte molecules (e.g., protein, antigen) to be profiled at single cell resolution. In some embodiments, the barcoded detector barcodes 332 are analyzed to identify and/or quantify the corresponding analyte molecules in the sample. In some embodiments the analysis of the barcoded detector barcodes 332 comprises genotyping, qPCR, sequencing, or combinations thereof.

Detection of Analyte Molecules using a Surface Assay

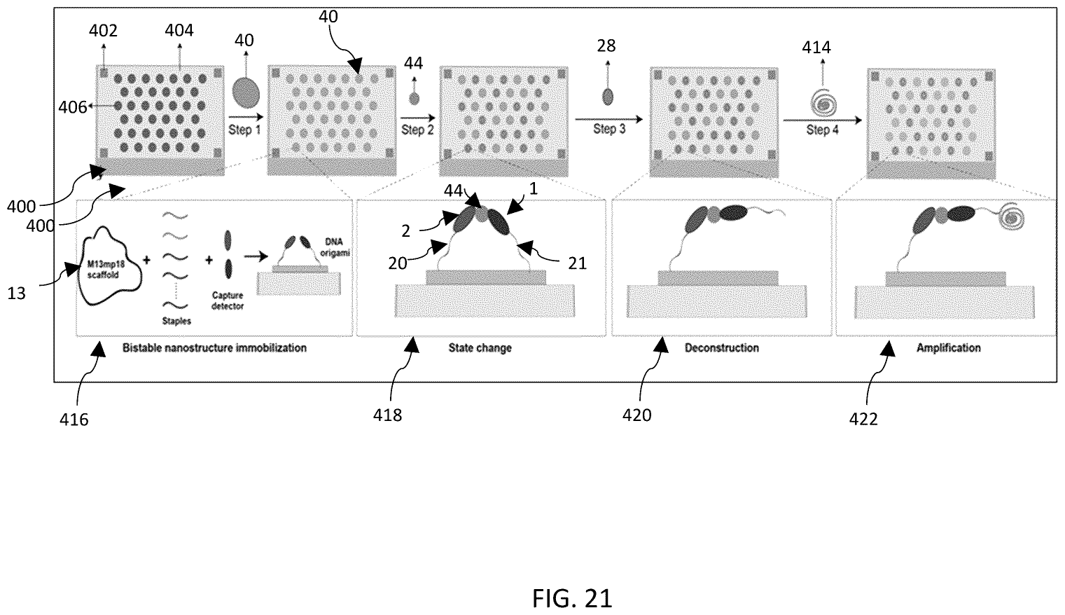

FIG. 21 provides an exemplary illustration of a method for detecting analyte molecules in a sample using a surface based assay that uses supramolecular structures, as described herein, for single-molecule counting of analytes in the sample (i.e. detecting analyte molecules in the sample at a single molecule resolution). In some embodiments, the supramolecular structures comprise a core structure comprising a DNA origami core. In some embodiments, a planar substrate 400 is provided comprising (a) Fiduciary markers 402 that serves as a reference coordinates for all the features on the substrate 400; (b) A defined set of micropatterned binding sites 406 where individual core structures (e.g., DNA origami) may be immobilized; (c) background passivation 404 that minimizes or prevents interaction between the surface of the substrate 400 and the supramolecular structure (including capture and detector molecules, core structure molecules). In some embodiments, the fiduciary markers comprise geometric features defined on a surface to be used as reference features for other features on the substrate. In some embodiments, the fiduciary markers 402 are coated with a polymer or self-assembled monolayer that does not interact with a core structure or other molecules of the supramolecular structure (e.g., DNA origami). In some embodiments, the background passivation 404 minimizes or prevents interaction between the surface of the substrate 400 and analyte molecules of the sample. In some embodiments, the planar substrate 400 comprises optical or electrical devices like FET, ring resonators, photonic crystals or microelectrode, to be defined prior to the formation of the binding sites 406. In some embodiments, the binding sites 406 are micropatterned on the planar substrate 400. In some embodiments, the binding sites 406 on the surface are in a periodic pattern. In some embodiments, the binding sites 406 on the surface are in a non-periodic pattern (e.g., random). In some embodiments, a minimum distance is specified between any two binding sites 406. In some embodiments, the minimum distance between any two binding sites 406 is at least about 200 nm. In some embodiments, the minimum distance between any two binding sites 406 is from at least about 40 nm to about 5000 nm. In some embodiments, the geometric shape of the binding sites 406 comprises a circle, square, triangle or other polygon shapes. In some embodiments, the chemical groups that are used for passivation 404 comprise neutrally charged molecules like a Tri-methyl silyl (TMS), an uncharged polymer like PEG, a zwitterionic polymer, or combinations thereof. In some embodiments, the chemical group used to define the binding site 406 comprises a silanol group, carboxyl group, thiol, other groups, or combinations thereof.

In some embodiments, a single supramolecular structure 40 is attached to a respective binding site 406 (Step 1). Reference character 416 provides a depiction of the components of the supramolecular structure 40, individually and as assembled and arranged on the planar substrate (components are as described herein, e.g., FIGS. 1, 2-3, 5-6). In some embodiments, the supramolecular structure 40 comprises a core structure 13 comprising a DNA origami, wherein the supramolecular structures 40 is attached onto each of the binding sites using DNA origami placement technique (step 1). In some embodiments, the supramolecular structure 40 is assembled prior to being attached to a respective binding site 406. In some embodiments, the DNA origami comprises a unique shape and dimension, so as to facilitate binding to a binding site using the DNA origami placement technique. In some embodiments, DNA origami placement comprises a directed self-assembly technique for organizing individual DNA origami (e.g., a core structure) on a surface (e.g., micropatterned surface). In some embodiments, alternatively to the DNA origami placement, a reactive group of the supramolecular nanostructure 40 is bound to a DNA origami that has been pre-organized on the binding site. In some embodiments, both of these methods for binding a supramolecular nanostructure to a corresponding binding site rely on the ability to organize one or more molecules on a micropatterned binding site using the DNA origami placement technique. In some embodiments, the planar substrate could be stored for a significant period after this step, in a clean environment.

With continued reference to FIG. 21, in some embodiments, a sample (as described herein) comprising analyte molecules is contacted with the planar substrate (step 2). In some embodiments, the sample is contacted with the planar substrate using a flow-cell. In some embodiments, the sample is incubated on the planar substrate with the supramolecular structures attached to the binding sites 406. In some embodiments, the incubation period may be from about 30 seconds to about 24 hours. In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

In some embodiments, the analyte molecules 44, in the sample, interact with the supramolecular structures 40 on the planar surface 400. In some embodiments, a single copy of a specific analyte molecule 44 binds simultaneously with both the capture and detector molecules, such that the particular supramolecular structure switches from an unstable state to a stable state 418 (as described herein, e.g., FIGS. 8-10). In some embodiments, a single copy of a particular analyte might interact simultaneously with the capture and detector molecules that are already bound to each other and switch the supramolecular structure from a stable state to an unstable state (as described herein, e.g., FIG. 11).

With continued reference to FIG. 21, in some embodiments, the planar substrate is then subjected to a trigger. In some embodiments, the trigger comprises a deconstructor molecule (e.g., detector deconstructor molecule 28 in FIG. 7). In some embodiments, the trigger comprises a trigger signal. In some embodiments, as described herein, the deconstructor molecule (e.g. detector deconstructor molecule 28) comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, deconstructor molecules associated with the supramolecular structures attached to the planar substrate is allowed to interact with said supramolecular structures. In some embodiments, the deconstructor molecules are introduced into the flow-cell containing the planar substrate. In some embodiments, the deconstructor molecule is incubated with the supramolecular structures from about 30 seconds to about 24 hours (step 3). In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, or from about 24 hours to about 48 hours.

In some embodiments, interaction with the deconstructor molecule cleaves the detector molecules and detector barcodes of all the supramolecular structures in the unstable state, such that these detector molecules and detector barcodes will be physically cleaved from the planar substrate 400. In some embodiments, the physically cleaved detector molecules and detector barcodes are removed during one or more buffer washes at the end of the incubation step. In some embodiments, wherein supramolecular structures on the planar substrate had shifted to a stable state, due to the capture of single analyte molecules, the corresponding detector molecules and detector barcodes are still linked to the supramolecular structure 420, and thereby stably bound to the planar substrate due to the analyte mediated sandwich formed between the corresponding detector and capture molecules (i.e. linkage between the capture molecule, analyte molecule, and detector molecule).

With continued reference to FIG. 21, in some embodiments, the detector barcode at the location of supramolecular structure that shifted to a stable state is used as a binding site 422 for a signaling element 414 (step 4). In some embodiments, the signaling element comprises a fluorescent molecule or microbead, a fluorescent polymer, highly charged nanoparticles or polymer. In some embodiments, one or more signaling elements are allowed to interact with the supramolecular structures on the planar structure. In some embodiments, the signaling elements are introduced into the flow-cell containing the planar substrates. In some embodiments, the detector barcode is used as a polymerization initiator for growth of highly fluorescent polymer in a process such as rolling circle amplification or hybridization chain reaction.

In some embodiments, introduction of the signaling element 414 as described with step 4 leads to a surface in which every individual analyte capture event (i.e. linkage between the capture molecule, detector molecule, and analyte molecule) leads to a signaling element being present at the location of the respective analyte (as linked with the capture and detector molecules). In some embodiments, the signaling element is optically active and can be measured using a microscope or integrated optically sensor within the planar substrate 400. In some embodiments, the signaling element is electrically active and may be measured using an integrated electrical sensor. In some embodiments, the signaling element is magnetically active and may be measured using an integrated magnetic sensor. In some embodiments, each signal event is associated with the capture of the same type of analyte molecule (a single copy of the same type of analyte molecule), determined by the corresponding detector and capture molecule, thus counting the number of locations where the signaling element is present gives the quantification of the analyte molecule in the sample.

In some embodiments, the method for detecting an analyte as described in FIG. 21 uses a supramolecular core wherein the core structure is bound to a DNA origami already organized on the surface of the planar substrate through a respective anchor moiety of the core structure.

In some embodiments, the method for detecting an analyte as described in FIG. 21 enables the detection of a single type of analyte molecule. In some embodiments, the method for detecting an analyte as described in FIG. 21 enables detection of a plurality of types of analyte molecules (multiplexed analyte molecule detection). In some embodiments, each supramolecular structure is barcoded to uniquely identify the respective capture and detector molecules associated, thereby enabling the respective analyte molecule captured to be identified. In some embodiments, each supramolecular structure is barcoded using the respective anchor molecule.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example

### Demonstration of DNA Based-Widget

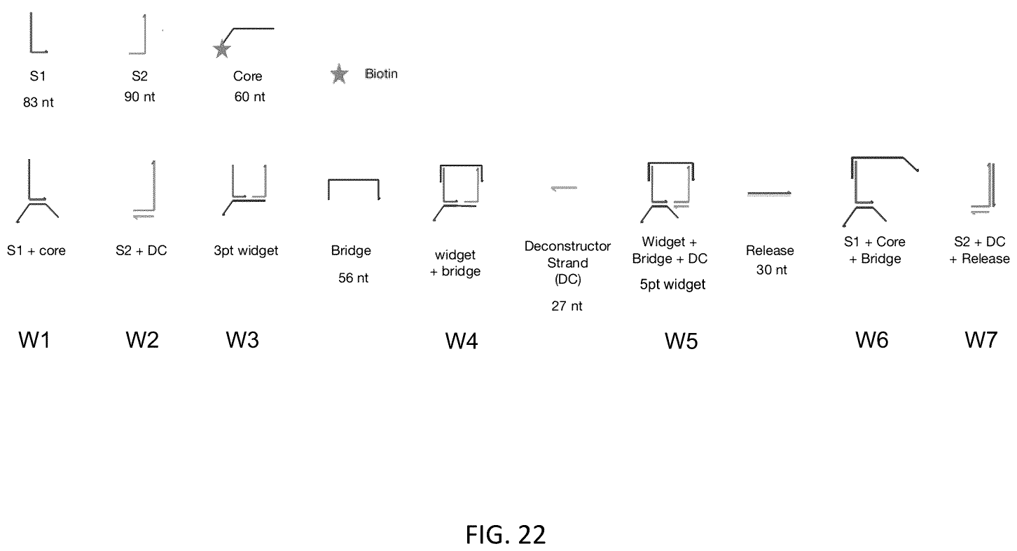

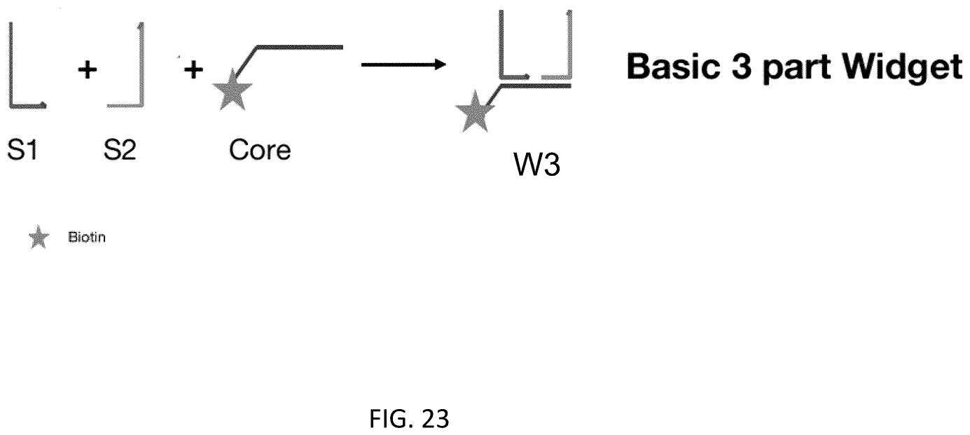

FIGS. 22-25 depict an exemplary process for evaluating the efficiency of a Deconstructor molecule, wherein a Bridge strand (as described herein) is used to mimic an antibody-antigen-antibody complex as described herein (for example, a capture molecule-analyte molecule-detector molecule complex). Change of a Widget structure upon the addition of a Deconstructor molecule is different for a Widget with a Bridge strand, compared to a Widget without a Bridge strand.

To evaluate the efficiency of a deconstructor, a three-part (3pt) Widget (W3) and a five-part (5pt) Widget (W5) were designed with various DNA strands. The three part-Widget included three single strands of DNA: S1, S2, and Core, wherein the Core is conjugated with biotin (FIGS. 22 & 23). The five part-Widget was constructed with five single strands of DNA: S1, S2, Core conjugated with biotin, Bridge, and Deconstructor (FIG. 22).

For the 3pt Widget (W3), the ability for the Widget to split into two sections of (S1+Core, W1) and S2 is vital for the proper functioning of the system. To the end, the strand displacement principles was used to utilize a short strand of DNA called the Deconstructor, or “DC” for short. The DC strand, when introduced to the 3pt Widget system, would displace the S2 strand. The objective of this experiment was to observe the efficiency of deconstruction of the 3pt Widget (W3) under simulated conditions with a “Bridge” strand that mimics the antibody-antigen-antibody complex that would be present in the final version of the Widget.

Since nuclease contamination for nucleic acid experiments can cause experimental inconsistency and even experimental failure, Nuclease Free deionized H₂O was used for all the procedures. The most commonly used buffer is Tris-EDTA (TE)-Mg buffer, and any buffer could be used as long as it contains enough cations to induce hybridization of DNA strands. A typical 1×TE-Mg buffer contains 10 mM Tris-HCl (pH 8.0), 1 mM EDTA, and 12.5 mM Mg²⁺.

Preparation of two different Widgets: All the DNA strands were suspended to 100 μM in H₂O. To make a 5pt Widget, 1 μl of each strand of five DNA parts (S1, S2, Core, Bridge, and DC) and 95 μL of TE-Mg buffer were added in a PCR tube to make a final volume of 100 μl and mixed by vortexing the tube for 10 seconds. To make a 3pt Widget, 1 μl of each strand of three DNA parts (S1, S2, and Core) and 97 μL of TE-Mg buffer were added in a PCR tube to make a final volume of 100 μl and mixed by vortexing the tube for 10 seconds. The tube was placed in thermocycler and the temperature of the thermocycler was programmed to run from 95° C. to 25° C. at a ramp of 1° C./min, with a final holding temperature of 4° C. The final product was stored at either 4° C. for a short term period or −20° C. for a long term period.

The DNA strands were suspended to 100 μM in H₂O. After the fabrication and purification of 3pt Widget, the concentration of 3pt Widget was measured and adjusted to 1.25 μM. The purified 3pt Widget, Bridge strand (except DC strands), 1×TE-Mg buffer (A, C, and E only) were mixed in six PCR tubes, following the recipes in Table x, by vortexing them and incubated for 1 hour at room temperature. After the 1 hr incubation, 1 μl of the 50 μM DC solution was added to tubes B, D, and F. The DNA mixtures in PCR tubes were by vortexing and further incubated for 30 minutes.

TABLE 1
Recipes for Deconstruction

| | | Volume (μM) of | | | Volume | Volume |
|---|---|---|---|---|---|---|
| | | 1.25 μM 3 pt | Volume (μM) of | | (μM) of 1x | (μM) of 50 |
| | | Widget | Bridge | | TE Mg | μM DC |
| A | Widget [1 μM] + 100 nM Bridge | 8 | 1 of 1 | μM | 1 | 0 |
| B | Widget [1 μM] + 100 nM Bridge + 5 μM DC | 8 | 1 of 1 | μM | 0 | 1 |
| C | Widget [1 μM] + 1 μM Bridge | 8 | 1 of 10 | μM | 1 | 0 |
| D | Widget [1 μM] + 1 μM Bridge + 5 μM | 8 | 1 of 10 | μM | 0 | 1 |
| E | Widget [1 μM] + 10 μM Bridge | 8 | 1 of 100 | μM | 1 | 0 |
| F | Widget [1 μM] + 10 μM Bridge + 5 μM DC | 8 | 1 of 100 | μM | 0 | 1 |

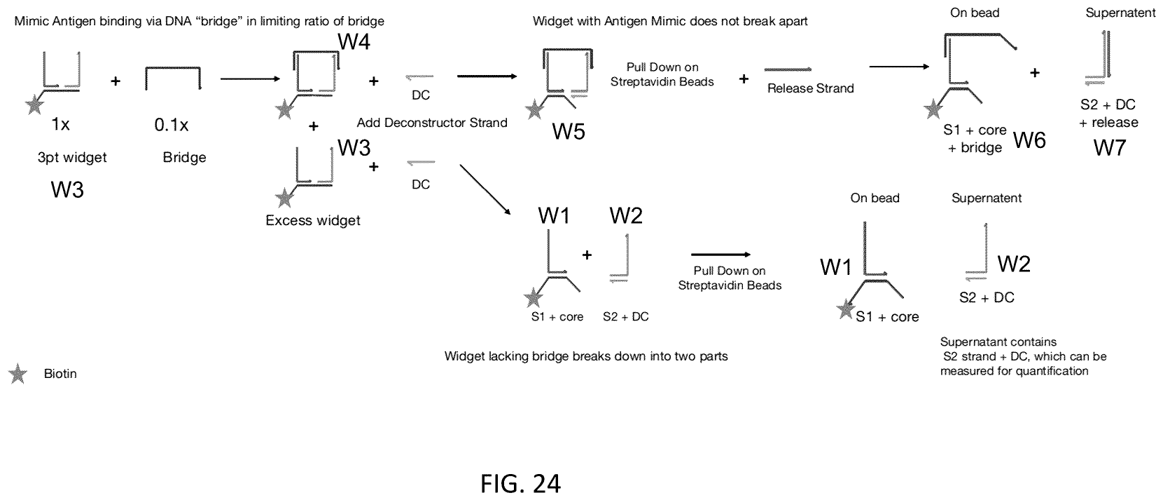

The working principle of the DNA based-Widget is illustrated in FIG. 24. The “Bridge” strand mimics an antigen binding to antibody. To clearly demonstrate the function of Bridge strand, a limited quantity of the Bridge strand was added to the purified 3pt Widget (W3) to make a final ratio of 3pt Widget/Bridge=1/0.1 (A case of Table 1). With limiting ratio (0.1 to 1) of Bridge strand compared to 3pt Widget (W3), ideally 10% of Widget would bind to the Bridge strand and the most of Widget would remain as a 3pt Widget (W3) itself. When DC strand is added, the Widget bound with Bridge (W4) does not break down while the Widget lacking Bridge (W3) breaks down into two parts including S1+Core (W1) and S2+DC (W2). With addition of streptavidin beads, the Widget with Bridge (W5) and S1+Core (W1) complexes are pulled down to the bottom of the solution, using strong interaction of Core-biotin and streptavidin beads, while the supernatant contains S2+DC (S2) complex which is readily separated from Widget lacking Bridge (W3). Further addition of a Release strand which is partially complementary to S2 strand enables the separation of S2+DC+Release (W7) complex from the complex of S1+Core+Bridge (W6) attached on the beads.

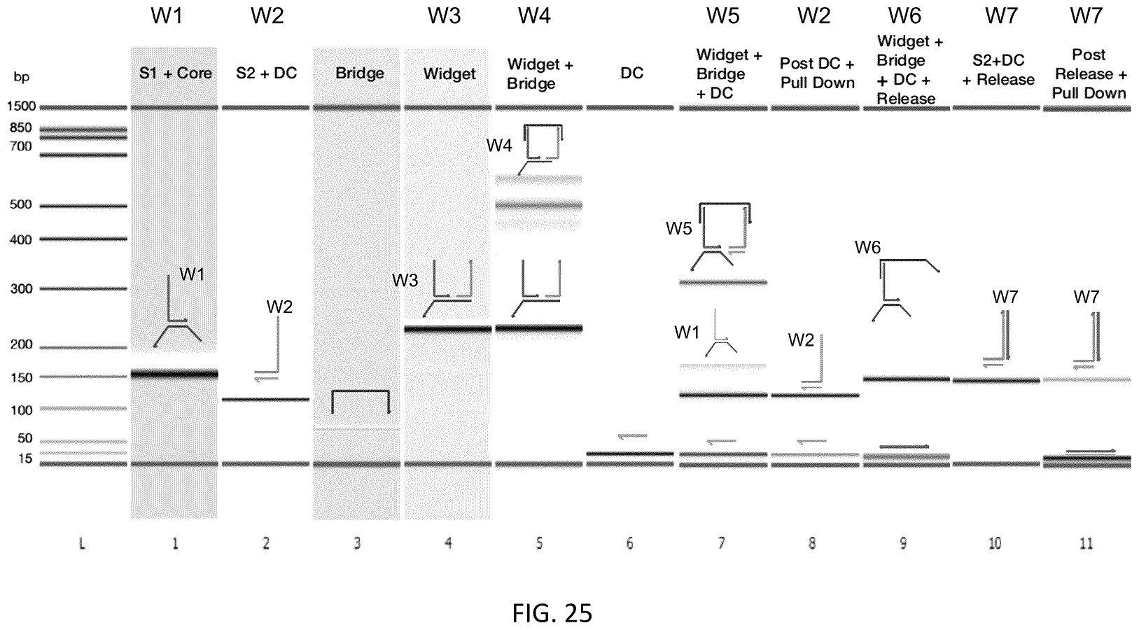

The efficiency of Deconstructor with DNA based-Widget was evaluated by agarose-gel electrophoresis (FIG. 25) of the molecular assemblies (W1-W7) as illustrated in FIG. 24. On the agarose gel, a strong band indicated an enrichment of the properly folded structures. As shown in FIG. 25, on the agarose gel, one band showed up for each W1 (Lane 1), W2 (Lane 2), and Bridge (Lane 3). The agarose gel showed that 3pt Widget was fabricated by a strong single band (Lane 4). When limiting amount of Bridge strand was added to W3, several bands appeared on the agarose gel, including a band corresponding to W3 and one strong band and minors of W4 (Lane 5), which showed a good agreement with the illustration of FIG. 24. Since the ratio of Bridge to W3 was 1/10, the most of W3 remained itself without binding with the Bridge strand. The several bands of W4 showed up because of intermolecular interactions between the Bridge and more than one W3 complexes. This interaction would be reduced later by the optimization of experimental conditions. Upon the addition of DC, W4 became W5 and W3 separated to two sections including W1 and W2 as shown in Lane 7. After both W5 and W1 containing biotin were pulled down to the tube upon the addition of streptavidin bead, the supernatant showed a band corresponding to W2 which was separated from W1 (originally W3) and a band of excess DC (Lane 8). When excess Release strand was further added, two bands corresponding to each W7 (identified as a Lane 10) and excess Release showed up with very light bands of W6 which mainly stayed with streptavidin bead in the bottom of the tube (Lane 9). The results of the agarose gel electrophoresis agreed well with the basic principle illustrated in FIG. 24 to demonstrate the efficiency of Deconstructor with DNA based-Widget.

1-209. (canceled)

210. A method for detecting an analyte molecule present in a sample, the method comprising:
(a) providing a supramolecular structure comprising:
i. a core structure comprising a plurality of core molecules,
ii. a capture molecule linked to the core structure at a first location, and
iii. a detector molecule linked to the core structure at a second location, wherein the supramolecular structure is in an unstable state, such that the detector molecule is configured to be unbound from the core structure through cleavage of a link therebetween at the second location;
(b) contacting the sample with the supramolecular structure, such that the supramolecular structure shifts from the unstable state to a stable state wherein the detector molecule and the capture molecule are linked together through binding to the analyte molecule, thereby forming a link between the detector molecule and capture molecule;
(c) providing a trigger to cleave the link between the detector molecule and the core structure at the second location, wherein the detector molecule remains linked to the core structure through the link with the capture molecule; and
(d) detecting the analyte molecule based on a signal provided by the supramolecular structure that shifted to the stable state.

211. The method of claim 210, wherein the analyte molecule comprises a protein, a peptide, a peptide fragment, a lipid, a DNA, a RNA, an organic molecule, an inorganic molecule, complexes thereof, or any combinations thereof.

212. The method claim 210, wherein the plurality of core molecules for each core structure are arranged into a pre-defined shape or have a prescribed molecular weight.

213. The method of claim 210, wherein the plurality of core molecules for each core structure comprises one or more nucleic acid strands, one or more branched nucleic acids, one or more peptides, one or more small molecules, or a combination thereof.

214. The method of claim 213, wherein each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multistranded DNA tile structure, a single-stranded RNA origami, a multi-stranded RNA tile structure, hierarchically composed DNA or RNA origami with multiple scaffolds, a peptide structure, or a combination thereof.

215. The method of claim 210, wherein the trigger comprises a deconstructor molecule, a trigger signal, or a combination thereof.

216. The method of claim 210, where in the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or a combination thereof.

217. The method of claim 210, wherein for each supramolecular structure:
(a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and
(b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker.

218. The method of claim 217, wherein the first core linker, second core linker, first capture linker, second capture linker, third capture linker, first detector linker, second detector linker, and third detector linker independently comprise a reactive molecule or DNA sequence domain.

219. The method of claim 210, wherein each supramolecular structure in the unstable state comprises the respective capture molecule and detector molecule spaced apart at a predetermined distance, so as to reduce or inhibit the occurrence of cross-reactions between capture and detector molecules of a first supramolecular structure and corresponding capture and detector molecules of a second supramolecular structure.

220. The method of claim 219, wherein the predetermined distance is from about 3 nm to about 40 nm.

221. The method of claim 210, wherein a plurality of analyte molecules in the sample are detected simultaneously through multiplexing via one or more supramolecular structures that shifted to a stable state.

222. The method of claim 210, wherein the capture and detector molecules for each supramolecular structure is configured for binding to one or more specific types of analyte molecules.

223. A substrate for detecting one or more analyte molecules in a sample, the substrate comprising a plurality of supramolecular structures, each supramolecular structure comprising:
(a) a core comprising a plurality of core molecules,
(b) a capture molecule linked to the core at a first location, and
(c) a detector molecule linked to the core at a second location,
wherein the supramolecular structure is in an unstable state, such that the detector molecule is configured to be unbound from the core through cleavage of a link therebetween at the second location;
wherein each supramolecular structure is configured to shift from the unstable state to a stable state through interaction between the detector molecule, the capture molecule, and a respective analyte molecule of the one or more analyte molecules; and
wherein, upon interaction with a trigger, a respective supramolecular structure that shifted to the stable state provides a signal for detecting the respective analyte molecule.

224. The substrate of claim 223, comprising a solid support, solid substrate, a polymer matrix, or a molecular condensate.

225. The substrate of claim 223, wherein the one or more analyte molecules comprises a protein, a peptide, a peptide fragment, a lipid, a DNA, a RNA, an organic molecule, an inorganic molecule, complexes thereof, or any combination thereof.

226. The substrate of claim 223, wherein the plurality of core molecules for each core structure are arranged into a pre-defined shape or have a prescribed molecular weight.

227. The substrate of claim 223, wherein each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multistranded DNA tile structure, a single-stranded RNA origami, a multi-stranded RNA tile structure, hierarchically composed DNA or RNA origami with multiple scaffolds, a peptide structure, or a combination thereof.

228. The substrate of claim 223, wherein the trigger comprises a deconstructor molecule, a trigger signal, or a combination thereof.

229. The substrate of claim 223, wherein the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or a combination thereof.

230. The substrate of claim 223, wherein for each supramolecular structure:
(a) the capture molecule is linked to the core through a capture barcode, wherein the capture barcode comprises a first capture linker, a second capture linker, and a capture bridge disposed between the first and second capture linkers, wherein the first capture linker is bound to a first core linker that is bound to the first location on the core, wherein the capture molecule and the second capture linker are linked together through binding to a third capture linker, and
(b) the detector molecule is linked to the core through a detector barcode, wherein the detector barcode comprises a first detector linker, a second detector linker, and a detector bridge disposed between the first and second detector linkers, wherein the first detector linker is bound to a second core linker that is bound to the second location on the core, wherein the detector molecule and the second detector linker are linked together through binding to a third detector linker.

231. The substrate of claim 230, wherein the first core linker, second core linker, first capture linker, second capture linker, third capture linker, first detector linker, second detector linker, and third detector linker independently comprise a reactive molecule or DNA sequence domain.

232. The substrate of claim 230, wherein the signal comprises the detector barcode, the capture barcode, or a combination thereof, corresponding to a supramolecular structure that shifted to a stable state.

233. The substrate of claim 223, wherein each supramolecular structure in the unstable state comprises the respective capture molecule and detector molecule spaced apart at a predetermined distance, so as to reduce or inhibit the occurrence of cross-reactions between capture and detector molecules of a first supramolecular structure and corresponding capture and detector molecules of a second supramolecular structure.

234. The substrate of claim 233, wherein the predetermined distance is from about 3 nm to about 40 nm.
